SEQUENCE LISTING (184 sequences)

SEQ ID NO: 1 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 185 aa, Mus musculus
MAQQCFHSEYFDSLLHACKPCHLRCSNPPATCQPYCDPSVTSSVKGTYTVLWIFLGLTLVLSLALFTISFLLRKMNPEALKDEPQSPGQLDGSAQLDKADTELTRIRAGDDRIFPRSLEYTVEECTCEDCVKSKPKGDSDHFFPLPAMEEGATILVTTKTGDYGKSSVPTALQSVMGMEKPTHTR

SEQ ID NO: 3 - protein, 184 aa, Rattus norvegicus
MAQRCFHSEYFDSLLHACKPCRLRCSNPPAPCQPYCDPSMTSSVRGTYTVLWIFLGLTLVVCLALFTLSFLLRKMNPEALKDKPQSPGHLDGSVQLDKADTERTKSRAGDERILPRSLEYTVEECTCEDCIKSNPKGDSDHFFPLPAMEEGATILVTTKTGDYGKGMPSALQSVMGMEKPAHSR

SEQ ID NO: 4 - protein, 183 aa, Macaca fascicularis
MLQMARQCSQNEYFDSLLHDCKPCQLRCSSTPPLTCQRYCNASMTNSVKGMNAILWTCLGLSLIISLAVFVLTFLLRKMSSEPLKDEFKNTGSGLLGMANIDLEKGRTGDEIVLPRGLEYTVEECTCEDCIKNKPKVDSDHCFPLPAMEEGATILVTTKTNDYCNSLSAALSVTEIEKSISAR

SEQ ID NO: 5 - protein, 330 aa, synthetic construct [sequence omitted]

SEQ ID NO: 6 - protein, 330 aa, synthetic construct
  source 1..330: note = Heavy Chain Constant Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 326 aa, synthetic construct
  source 1..326: note = Heavy Chain Constant Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - skipped

SEQ ID NO: 9 - protein, 329 aa, synthetic construct
  source 1..329: note = Heavy Chain Constant Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 106 aa, synthetic construct
  source 1..106: note = Light Chain Constant Domain
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 120 aa, synthetic construct
  source 1..120: note = Variable heavy (vh) domain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRQWSSSWYFDYWGQGTLVTVSS

SEQ ID NO: 13 - protein, 5 aa, synthetic construct
  source 1..5: note = vhCDR1
SYAMS

SEQ ID NO: 14 - protein, 17 aa, synthetic construct
  source 1..17: note = vhCDR2
AISGSGGSTYYADSVKG

SEQ ID NO: 15 - protein, 11 aa, synthetic construct
  source 1..11: note = vhCDR3
RQWSSSWYFDY

SEQ ID NO: 16 - protein, 107 aa, synthetic construct
  source 1..107: note = Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKLEIK

SEQ ID NO: 17 - protein, 11 aa, synthetic construct
  source 1..11: note = vlCDR1
RASQSISSYLN

SEQ ID NO: 18 - protein, 7 aa, synthetic construct
  source 1..7: note = vlCDR2
AASSLQS

SEQ ID NO: 19 - protein, 9 aa, synthetic construct
  source 1..9: note = vlCDR3
QQSYSTPPT

SEQ ID NO: 20 - protein, 449 aa, synthetic construct
  source 1..449: note = Heavy Chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRQWSSSWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 214 aa, synthetic construct
  source 1..214: note = Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 120 aa, synthetic construct
  source 1..120: note = Variable heavy (vh) domain
EVQLLESGGGLVQPGGSLRLSCAASGFTFSSYAMSWVRQAPGKGLEWVSAISGSGDSTFYADSVKGRFTISRDNSKNTLYLQMNSLRAEDTAVYYCAKRRWGRTWYFDYWGQGTLVTVSS

SEQ ID NO: 23 - protein, 5 aa, synthetic construct
SYAMS

SEQ ID NO: 24 - protein, 17 aa, synthetic construct
  source 1..17: note = vhCDR2
AISGSGDSTFYADSVKG

SEQ ID NO: 25 - protein, 11 aa, synthetic construct
RRWGRTWYFDY

SEQ ID NO: 26 - protein, 107 aa, synthetic construct; identical to SEQ ID NO: 16

SEQ ID NO: 27 - protein, 11 aa, synthetic construct
RASQSISSYLN

SEQ ID NO: 28 - protein, 7 aa, synthetic construct
AASSLQS

SEQ ID NO: 29 - protein, 9 aa, synthetic construct
QQSYSTPPT

SEQ ID NO: 30 - protein, 449 aa, synthetic construct
EVQLLESGGGLVQPGGSLRLSCAASGFTFSSYAMSWVRQAPGKGLEWVSAISGSGDSTFYADSVKGRFTISRDNSKNTLYLQMNSLRAEDTAVYYCAKRRWGRTWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, synthetic construct; identical to SEQ ID NO: 21

SEQ ID NO: 32 - protein, 121 aa, synthetic construct
EVQLLESGGGLVQPGGSLRLSCAASGFTFSNYDMAWVRQAPGKGLEWVSSISRSGDSTLYRDSVKGRFTISRDNSKSTLYLQMNSLRAEDTAVYYCASQNYGGFGDWFAWWGQGTLVTVSS

SEQ ID NO: 33 - protein, 5 aa, synthetic construct
NYDMA

SEQ ID NO: 34 - protein, 17 aa, synthetic construct
SISRSGDSTLYRDSVKG

SEQ ID NO: 35 - protein, 12 aa, synthetic construct
QNYGGFGDWFAW

SEQ ID NO: 36 - protein, 107 aa, synthetic construct
DTVLTQSPSSLSASVGDRVTITCRASEGVSTLMHWYQQKPGKQPKLLIYLASHLESGVPARFSGSGSGTDFTLTISSLQPEDFATYYCQQSWNDPRTFGGGTKVEIK

SEQ ID NO: 37 - protein, 11 aa, synthetic construct
RASEGVSTLMH

SEQ ID NO: 38 - protein, 7 aa, synthetic construct
LASHLES

SEQ ID NO: 39 - protein, 9 aa, synthetic construct
QQSWNDPRT

SEQ ID NO: 40 - protein, 450 aa, synthetic construct
EVQLLESGGGLVQPGGSLRLSCAASGFTFSNYDMAWVRQAPGKGLEWVSSISRSGDSTLYRDSVKGRFTISRDNSKSTLYLQMNSLRAEDTAVYYCASQNYGGFGDWFAWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, synthetic construct
DTVLTQSPSSLSASVGDRVTITCRASEGVSTLMHWYQQKPGKQPKLLIYLASHLESGVPARFSGSGSGTDFTLTISSLQPEDFATYYCQQSWNDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 121 aa, synthetic construct
EVQLLESGGGLVQPGGSLRLSCAASGFIFSNYDMAWVRQAPGKGLEWVSSISRSGGSTSYRDSVKGRFTISRDNSKSTLYLQMNSLRAEDTAVYYCARLDYGYNYAWIAYWGQGTLVTVSS

SEQ ID NO: 43 - protein, 5 aa, synthetic construct
NYDMA

SEQ ID NO: 44 - protein, 17 aa, synthetic construct
SISRSGGSTSYRDSVKG

SEQ ID NO: 45 - skipped

SEQ ID NO: 46 - protein, 107 aa, synthetic construct
DIQMTQSPSSLSASVGDRVTITCRASEGISNDLAWYQQKPGKSPKLLIYAASRLQDGVPSRFSGSGSGTDYTLTISSLQPEDFATYFCQQSYKYPFTFGSGTKLEIK

SEQ ID NO: 47 - protein, 11 aa, synthetic construct
RASEGISNDLA

SEQ ID NO: 48 - protein, 7 aa, synthetic construct
AASRLQD

SEQ ID NO: 49 - protein, 9 aa, synthetic construct
QQSYKYPFT

SEQ ID NO: 50 - protein, 450 aa, synthetic construct
EVQLLESGGGLVQPGGSLRLSCAASGFIFSNYDMAWVRQAPGKGLEWVSSISRSGGSTSYRDSVKGRFTISRDNSKSTLYLQMNSLRAEDTAVYYCARLDYGYNYAWIAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 214 aa, synthetic construct
DIQMTQSPSSLSASVGDRVTITCRASEGISNDLAWYQQKPGKSPKLLIYAASRLQDGVPSRFSGSGSGTDYTLTISSLQPEDFATYFCQQSYKYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 121 aa, synthetic construct
EVQLLESGGGLVQPGGSLRLSCAASGFTFSNYDMAWVRQAPGKGLEWVSSISTSGDITYYRDSVKGRFTISRDNSKSTLYLQMNSLRAEDTAVYYCARQGYYDGSYLFDYWGQGTLVTVSS

SEQ ID NO: 53 - protein, 5 aa, synthetic construct
NYDMA

SEQ ID NO: 54 - protein, 17 aa, synthetic construct
SISTSGDITYYRDSVKG

SEQ ID NO: 55 - protein, 12 aa, synthetic construct
QGYYDGSYLFDY

SEQ ID NO: 56 - protein, 107 aa, synthetic construct
DIQMTQSPSSLSASVGDRVTITCRASQGISNNLNWYQQKPGKTIKPLIYYTSNLQSGVPSRFSGSRSGTDYTLTISSLQPEDFATYYCQQVASFPYTFGAGTKLEIK

SEQ ID NO: 57 - protein, 11 aa, synthetic construct
RASQGISNNLN

SEQ ID NO: 58 - protein, 7 aa, synthetic construct
YTSNLQS

SEQ ID NO: 59 - protein, 9 aa, synthetic construct
QQVASFPYT

SEQ ID NO: 60 - protein, 450 aa, synthetic construct
EVQLLESGGGLVQPGGSLRLSCAASGFTFSNYDMAWVRQAPGKGLEWVSSISTSGDITYYRDSVKGRFTISRDNSKSTLYLQMNSLRAEDTAVYYCARQGYYDGSYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 214 aa, synthetic construct
DIQMTQSPSSLSASVGDRVTITCRASQGISNNLNWYQQKPGKTIKPLIYYTSNLQSGVPSRFSGSRSGTDYTLTISSLQPEDFATYYCQQVASFPYTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 121 aa, synthetic construct
EVQLLESGGGLVQPGGSLRLSCAASGFTFSNYDMAWVRQAPGKGLEWVSSISTSGDTTSYRDSVKGRFTISRDNSKSTLYLQMNSLRAEDTAVYYCARQDSSYMGDWFAYWGQGTLVTVSS

SEQ ID NO: 63 - protein, 5 aa, synthetic construct
NYDMA

SEQ ID NO: 64 - protein, 17 aa, synthetic construct
SISTSGDTTSYRDSVKG

SEQ ID NO: 65 - protein, 12 aa, synthetic construct
QDSSYMGDWFAY

SEQ ID NO: 66 - protein, 107 aa, synthetic construct
DIVMTQSPLSLPVTPGEPASISCRASQNVGSNVDWFLQKPGQSPQLLIYTASNRYTGVPDRFTGSGSGTDFTLKISRVEAEDVGVYYCMQSNSYPLTFGSGTKLEIK

SEQ ID NO: 67 - protein, 11 aa, synthetic construct
RASQNVGSNVD

SEQ ID NO: 68 - protein, 7 aa, synthetic construct
TASNRYT

SEQ ID NO: 69 - protein, 9 aa, synthetic construct
MQSNSYPLT

SEQ ID NO: 70 - protein, 450 aa, synthetic construct
EVQLLESGGGLVQPGGSLRLSCAASGFTFSNYDMAWVRQAPGKGLEWVSSISTSGDTTSYRDSVKGRFTISRDNSKSTLYLQMNSLRAEDTAVYYCARQDSSYMGDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 214 aa, synthetic construct
DIVMTQSPLSLPVTPGEPASISCRASQNVGSNVDWFLQKPGQSPQLLIYTASNRYTGVPDRFTGSGSGTDFTLKISRVEAEDVGVYYCMQSNSYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 122 aa, synthetic construct
EVQLLESGGGLVQPGGSLRLSCAASGFIFSNYDMAWVRQAPGKGLEWVSSISTSGDITNYRDSVKGRFTISRDNSKITLYLQMNSLRAEDTAVYFCARQAYYGYNAGVMDVWGQGTTVTVSS

SEQ ID NO: 73 - protein, 5 aa, synthetic construct
NYDMA

SEQ ID NO: 74 - protein, 17 aa, synthetic construct
SISTSGDITNYRDSVKG

SEQ ID NO: 75 - protein, 13 aa, synthetic construct
QAYYGYNAGVMDV

SEQ ID NO: 76 - protein, 107 aa, synthetic construct
DVQMTQSPSSLSASVGDRVTITCRASKSISKYLAWYQQKPGKANKLLIYSGSTLQSGTPSRFSGSGSGTDFTLTISSLQPEDFATYYCQQYNEYPFTFGSGTKLEIK

SEQ ID NO: 77 - protein, 11 aa, synthetic construct
RASKSISKYLA

SEQ ID NO: 78 - protein, 7 aa, synthetic construct
SGSTLQS

SEQ ID NO: 79 - protein, 9 aa, synthetic construct
QQYNEYPFT

SEQ ID NO: 80 - protein, 451 aa, synthetic construct
EVQLLESGGGLVQPGGSLRLSCAASGFIFSNYDMAWVRQAPGKGLEWVSSISTSGDITNYRDSVKGRFTISRDNSKITLYLQMNSLRAEDTAVYFCARQAYYGYNAGVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 214 aa, synthetic construct
DVQMTQSPSSLSASVGDRVTITCRASKSISKYLAWYQQKPGKANKLLIYSGSTLQSGTPSRFSGSGSGTDFTLTISSLQPEDFATYYCQQYNEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 15 aa, synthetic construct
GGGGSGGGGSGGGGS

SEQ ID NO: 83 - protein, 18 aa, synthetic construct
GSTSGSGKPGSGEGSTKG

SEQ ID NO: 84 - protein, 14 aa, synthetic construct
IRPRAIGGSKPRVA

SEQ ID NO: 85 - protein, 15 aa, synthetic construct
GKGGSGKGGSGKGGS

SEQ ID NO: 86 - protein, 15 aa, synthetic construct
GGKGSGGKGSGGKGS

SEQ ID NO: 87 - protein, 15 aa, synthetic construct
GGGKSGGGKSGGGKS

SEQ ID NO: 88 - protein, 15 aa, synthetic construct
GKGKSGKGKSGKGKS

SEQ ID NO: 89 - protein, 15 aa, synthetic construct
GGGKSGGKGSGKGGS

SEQ ID NO: 90 - protein, 15 aa, synthetic construct
GKPGSGKPGSGKPGS

SEQ ID NO: 91 - protein, 20 aa, synthetic construct
GKPGSGKPGSGKPGSGKPGS

SEQ ID NO: 92 - protein, 20 aa, synthetic construct
GKGKSGKGKSGKGKSGKGKS

SEQ ID NO: 93 - protein, 20 aa, synthetic construct
GGGGSGGGGSGGGGSGGGGS

SEQ ID NO: 94 - protein, 14 aa, synthetic construct
STAGDTHLGGEDFD

SEQ ID NO: 95 - protein, 15 aa, synthetic construct
GEGGSGEGGSGEGGS

SEQ ID NO: 96 - protein, 15 aa, synthetic construct
GGEGSGGEGSGGEGS

SEQ ID NO: 97 - protein, 15 aa, synthetic construct
GGGESGGGESGGGES

SEQ ID NO: 98 - protein, 15 aa, synthetic construct
GEGESGEGESGEGES

SEQ ID NO: 99 - protein, 15 aa, synthetic construct
GGGESGGEGSGEGGS

SEQ ID NO: 100 - protein, 20 aa, synthetic construct
GEGESGEGESGEGESGEGES

SEQ ID NO: 101 - protein, 15 aa, synthetic construct
GGGGSGGGGSGGGGS

SEQ ID NO: 102 - protein, 20 aa, synthetic construct
GGGGSGGGGSGGGGSGGGGS

SEQ ID NO: 103 - protein, 18 aa, synthetic construct
GSTSGSGKPGSGEGSTKG

SEQ ID NO: 104 - protein, 19 aa, synthetic construct
PRGASKSGSASQTGSAPGS

SEQ ID NO: 105 - protein, 19 aa, synthetic construct
GTAAAGAGAAGGAAAGAAG

SEQ ID NO: 106 - protein, 19 aa, synthetic construct
GTSGSSGSGSGGSGSGGGG

SEQ ID NO: 107 - protein, 20 aa, synthetic construct
GKPGSGKPGSGKPGSGKPGS

SEQ ID NO: 108 - protein, 5 aa, synthetic construct
GGGGS

SEQ ID NO: 109 - protein, 10 aa, synthetic construct
GGGGSGGGGS

SEQ ID NO: 110 - protein, 15 aa, synthetic construct
GGGGSGGGGSGGGGS

SEQ ID NO: 111 - protein, 20 aa, synthetic construct
GGGGSGGGGSGGGGSGGGGS

SEQ ID NO: 112 - protein, 25 aa, synthetic construct
GGGGSGGGGSGGGGSGGGGSGGGGS

SEQ ID NO: 113 - protein, 30 aa, synthetic construct
GGGGSGGGGSGGGGSGGGGSGGGGSGGGGS

SEQ ID NO: 114 - protein, 35 aa, synthetic construct
GGGGSGGGGSGGGGSGGGGSGGGGSGGGGSGGGGS

SEQ ID NO: 115 - protein, 5 aa, synthetic construct
GGGGA

SEQ ID NO: 116 - protein, 10 aa, synthetic construct
GGGGAGGGGA

SEQ ID NO: 117 - protein, 15 aa, synthetic construct
GGGGAGGGGAGGGGA

SEQ ID NO: 118 - protein, 20 aa, synthetic construct
GGGGAGGGGAGGGGAGGGGA

SEQ ID NO: 119 - protein, 25 aa, synthetic construct
GGGGAGGGGAGGGGAGGGGAGGGGA

SEQ ID NO: 120 - protein, 30 aa, synthetic construct
GGGGAGGGGAGGGGAGGGGAGGGGAGGGGA

SEQ ID NO: 121 - protein, 35 aa, synthetic construct
GGGGAGGGGAGGGGAGGGGAGGGGAGGGGAGGGGA

SEQ ID NO: 122 - protein, 30 aa, synthetic construct
DPALVHQRPAPPGGGGSGGGGSGGGGSGGG

SEQ ID NO: 123 - protein, 5 aa, synthetic construct
GKPGS

SEQ ID NO: 124 - protein, 25 aa, synthetic construct
GKPGSGKPGSGKPGSGKPGSGKPGS

SEQ ID NO: 125 - protein, 30 aa, synthetic construct
GKPGSGKPGSGKPGSGKPGSGKPGSGKPGS

SEQ ID NO: 126 - protein, 5 aa, synthetic construct
GGGES

SEQ ID NO: 127 - protein, 9 aa, synthetic construct
KTHTCPPCP

SEQ ID NO: 128 - protein, 15 aa, synthetic construct
EPKSSDKTHTCPPCP

SEQ ID NO: 129 - protein, 19 aa, synthetic construct
GGGGSGGGGSKTHTCPPCP

SEQ ID NO: 130 - protein, 19 aa, synthetic construct
GKPGSGKPGSKTHTCPPCP

SEQ ID NO: 131 - protein, 14 aa, synthetic construct
GKPGSKTHTCPPCP

SEQ ID NO: 132 - protein, 5 aa, synthetic construct
EPKSC

SEQ ID NO: 133 - protein, 15 aa, synthetic construct
EPKSCGGGGSGGGGS

SEQ ID NO: 134 - protein, 15 aa, synthetic construct
EPKSCGKPGSGKPGS

SEQ ID NO: 135 - protein, 10 aa, synthetic construct
EPKSCGKPGS

SEQ ID NO: 136 - protein, 216 aa, synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 137 - protein, 216 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 216 aa, synthetic construct; identical to SEQ ID NO: 136

SEQ ID NO: 139 - protein, 216 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 216 aa, synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 141 - protein, 216 aa, synthetic construct; identical to SEQ ID NO: 139

SEQ ID NO: 142 - protein, 216 aa, synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 143 - protein, 216 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 144 - protein, 216 aa, synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 145 - protein, 216 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 216 aa, synthetic construct
APPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 147 - protein, 216 aa, synthetic construct
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 216 aa, synthetic construct
APPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 149 - protein, 216 aa, synthetic construct
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 217 aa, synthetic construct
APEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 151 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 152 - protein, 216 aa, synthetic construct
APPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 153 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 154 - protein, 216 aa, synthetic construct
APPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 155 - protein, 216 aa, synthetic construct
APPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 156 - protein, 216 aa, synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 157 - protein, 216 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 158 - protein, 216 aa, synthetic construct
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 216 aa, synthetic construct; identical to SEQ ID NO: 136

SEQ ID NO: 160 - protein, 215 aa, synthetic construct
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPK

SEQ ID NO: 161 - protein, 214 aa, synthetic construct
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSP

SEQ ID NO: 162 - protein, 113 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 163 - protein, 113 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCP

SEQ ID NO: 164 - protein, 110 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 165 - protein, 110 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTVERKCCVECPPCP

SEQ ID NO: 166 - protein, 110 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCP

SEQ ID NO: 167 - protein, 110 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRVESKYGPPCPSCP

SEQ ID NO: 168 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 169 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSC

SEQ ID NO: 170 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 171 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKV

SEQ ID NO: 172 - protein, 98 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 173 - protein, 98 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTV

SEQ ID NO: 174 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 175 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRV

SEQ ID NO: 176 - protein, 15 aa, synthetic construct
EPKSCDKTHTCPPCP

SEQ ID NO: 177 - protein, 12 aa, synthetic construct
ERKCCVECPPCP

SEQ ID NO: 178 - protein, 12 aa, synthetic construct
ESKYGPPCPSCP

SEQ ID NO: 179 - protein, 107 aa, synthetic construct [sequence omitted]

SEQ ID NO: 180 - protein, 106 aa, synthetic construct; identical to SEQ ID NO: 11

SEQ ID NO: 181 - protein, 54 aa, Homo sapiens
MLQMAGQCSQNEYFDSLLHACIPCQLRCSSNTPPLTCQRYCNASVTNSVKGTNA

SEQ ID NO: 182 - protein, 53 aa, synthetic construct
MLQMARQCSQNEYFDSLLHDCKPCQLRCSSTPPLTCQRYCNASMTNSVKGMNA

SEQ ID NO: 183 - protein, 49 aa, Mus musculus
MAQQCFHSEYFDSLLHACKPCHLRCSNPPATCQPYCDPSVTSSVKGTYT

SEQ ID NO: 184 - protein, 49 aa, synthetic construct
MAQRCFHSEYFDSLLHACKPCRLRCSNPPAPCQPYCDPSMTSSVRGTYT

(19) United States
(12) Patent Application Publication
(10) Pub. No.: US 2024/0392025 A1
(43) Pub. Date: Nov. 28, 2024

(54) B-CELL MATURATION ANTIGEN (BCMA) BINDING DOMAIN COMPOSITIONS

(71) Applicant: Xencor, Inc., Pasadena, CA (US)

(72) Inventor: Gregory Moore, Azusa, CA (US)

(21) Appl. No.: 18/694,877

(22) PCT Filed: Sep. 27, 2022
(86) PCT No.: PCT/US2022/077070

§ 371 Date: Mar. 22, 2024

Related U.S. Application Data
(60) Provisional application No. 63/248,988, filed on Sep. 27, 2021.

(51) Int. Cl. C07K 16/28

(52) U.S. Cl. CPC C07K 16/2878; C07K 2317/565

(57) ABSTRACT

Provided herein are novel BCMA binding domain compositions and anti-BCMA antibodies that include such BCMA binding domains.

### PRIORITY CLAIM

This application claims priority to and benefit of U.S. Provisional Application No. 63/248,988, filed on Sep. 27, 2021, the contents of which are hereby incorporated by reference in their entirety.

### BACKGROUND

The instant application contains a Sequence Listing which has been submitted electronically in XML format and is hereby incorporated by reference in its entirety. Said XML copy, created on Sep. 26, 2022, is named 067461-5292-WO.xml and is 177,871 bytes in size.

### BACKGROUND

Antibody-based therapeutics have been used successfully to treat a variety of diseases.

B-cell maturation antigen (BCMA) is a member of the TNF receptor superfamily, which recognizes B-cell activating factor (BAFF). See, .e.g., Laabi et al., The EMBO Journal 11(11): 3897-904 (1992). BCMA is preferentially expressed in mature B lymphocytes and is thought to play an important role for B cell development and autoimmune response. In B cell development, BCMA plays a central role in regulating B cell maturation and differentiation into plasma cells by engaging a proliferation-inducing ligand (APRIL) and also is necessary for plasma cell survival in the bone marrow. See, e.g., Tai et al., Immunotherapy 7:1187-1199 (2015).

BCMA is implicated in various hematological cancers including, but not limited to, leukemia, lymphomas, and multiple myeloma. Moreaux et al., Blood 103:2148-3157 (2004); and Chiu et al., Blood 109 (2): 729-739 (2007). With respect to multiple myeloma, BCMA is expressed on multiple myeloma cell lines and malignant plasma cells and BCMA expression increases during multiple myeloma disease progression. See Moreaux et al., Blood 103:2148-3157 (2004); Carpenter et al., Clin Cancer Res 19:2048-2060 (2013); and Yong et al., Blood 122:4447 (2013). There remains a need for novel therapeutics for the treatment of hematological diseases, particularly those that target BCMA.

### SUMMARY

Provided herein are novel BCMA antigen binding domain compositions and antibodies that include such BCMA antigen binding domains. The BCMA antigen binding domains and antibodies provided herein are useful, for example, in the treatment of BCMA-associated diseases.

Provided herein are novel BCMA antigen binding domain compositions and antibodies that include such BCMA antigen binding domains. The BCMA antigen binding domains and antibodies provided herein are useful, for example, in the treatment of BCMA-associated diseases.

In one aspect provided herein is a composition comprising an anti-BCMA antigen binding domain comprising a set of a variable light domain and a variable heavy domain selected from the following sets of variable light domains and variable heavy domains: a) a variable light domain comprising a vlCDR1 having an amino acid of SEQ ID NO: 17, a vlCDR2 having an amino acid of SEQ ID NO: 18, and a vlCDR3 having an amino acid of SEQ ID NO: 19 and a variable heavy domain comprising a vhCDR1 having an amino acid of SEQ ID NO: 13, a vhCDR2 having an amino acid of SEQ ID NO: 14 and a vhCDR3 having an amino acid of SEQ ID NO: 15; b) a variable light domain comprising a vlCDR1 having an amino acid of SEQ ID NO: 27, a vlCDR2 having an amino acid of SEQ ID NO: 28, and a vlCDR3 having an amino acid of SEQ ID NO: 29 and a variable heavy domain comprising a vhCDR1 having an amino acid of SEQ ID NO: 23, a vhCDR2 having an amino acid of SEQ ID NO: 24 and a vhCDR3 having an amino acid of SEQ ID NO: 25; c) a variable light domain comprising a vlCDR1 having an amino acid of SEQ ID NO: 37, a vlCDR2 having an amino acid of SEQ ID NO: 38, and a vlCDR3 having an amino acid of SEQ ID NO: 39 and a variable heavy domain comprising a vhCDR1 having an amino acid of SEQ ID NO: 33, a vhCDR2 having an amino acid of SEQ ID NO: 34 and a vhCDR3 having an amino acid of SEQ ID NO: 35; d) a variable light domain comprising a vlCDR1 having an amino acid of SEQ ID NO: 47, a vlCDR2 having an amino acid of SEQ ID NO: 48, and a vlCDR3 having an amino acid of SEQ ID NO: 49 and a variable heavy domain comprising a vhCDR1 having an amino acid of SEQ ID NO: 43, a vhCDR2 having an amino acid of SEQ ID NO: 44 and a vhCDR3 having an amino acid of SEQ ID NO: 45; e) a variable light domain comprising a vlCDR1 having an amino acid of SEQ ID NO: 57, a vlCDR2 having an amino acid of SEQ ID NO: 58, and a vlCDR3 having an amino acid of SEQ ID NO: 59 and a variable heavy domain comprising a vhCDR1 having an amino acid of SEQ ID NO: 53, a vhCDR2 having an amino acid of SEQ ID NO: 54 and a vhCDR3 having an amino acid of SEQ ID NO: 55; f) a variable light domain comprising a vlCDR1 having an amino acid of SEQ ID NO: 67, a vlCDR2 having an amino acid of SEQ ID NO: 68, and a vlCDR3 having an amino acid of SEQ ID NO: 69 and a variable heavy domain comprising a vhCDR1 having an amino acid of SEQ ID NO: 63, a vhCDR2 having an amino acid of SEQ ID NO: 64 and a vhCDR3 having an amino acid of SEQ ID NO: 65; and g) a variable light domain comprising a vlCDR1 having an amino acid of SEQ ID NO: 77, a vlCDR2 having an amino acid of SEQ ID NO: 78, and a vlCDR3 having an amino acid of SEQ ID NO: 79 and a variable heavy domain comprising a vhCDR1 having an amino acid of SEQ ID NO: 73, a vhCDR2 having an amino acid of SEQ ID NO: 74 and a vhCDR3 having an amino acid of SEQ ID NO: 75.

In another aspect, provided herein is a composition comprising an anti-BCMA antigen binding domain comprising a set of a variable light domain and a variable heavy domain selected from the following sets of variable light domains and variable heavy domains: a) a variable light domain comprising a vlCDR1, vlCDR2, and vlCDR3 of a variable light domain having the amino acid of SEQ ID NO: 16 and a variable heavy domain comprising a vhCDR1, vhCDR2, and vhCDR3 of a variable heavy domain having the amino acid sequence of SEQ ID NO: 12; b) a variable light domain comprising a vlCDR1, vlCDR2, and vlCDR3 of a variable light domain having the amino acid of SEQ ID NO: 26 and a variable heavy domain comprising a vhCDR1, vhCDR2, and vhCDR3 of a variable heavy domain having the amino acid sequence of SEQ ID NO: 22; c) a variable light domain comprising a vlCDR1, vlCDR2, and vlCDR3 of a variable light domain having the amino acid of SEQ ID NO: 36 and a variable heavy domain comprising a vhCDR1, vhCDR2, and vhCDR3 of a variable heavy domain having the amino acid sequence of SEQ ID NO: 32; d) a variable light domain comprising a vlCDR1, vlCDR2, and vlCDR3 of a variable light domain having the amino acid of SEQ ID NO: 46 and a variable heavy domain comprising a vhCDR1, vhCDR2, and vhCDR3 of a variable heavy domain having the amino acid sequence of SEQ ID NO: 42; e) a variable light domain comprising a vlCDR1, vlCDR2, and vlCDR3 of a variable light domain having the amino acid of SEQ ID NO: 56 and a variable heavy domain comprising a vhCDR1, vhCDR2, and vhCDR3 of a variable heavy domain having the amino acid sequence of SEQ ID NO: 52; f) a variable light domain comprising a vlCDR1, vlCDR2, and vlCDR3 of a variable light domain having the amino acid of SEQ ID NO: 66 and a variable heavy domain comprising a vhCDR1, vhCDR2, and vhCDR3 of a variable heavy domain having the amino acid sequence of SEQ ID NO: 62; and g) a variable light domain comprising a vlCDR1, vlCDR2, and vlCDR3 of a variable light domain having the amino acid of SEQ ID NO: 76 and a variable heavy domain comprising a vhCDR1, vhCDR2, and vhCDR3 of a variable heavy domain having the amino acid sequence of SEQ ID NO: 72.

In one aspect, provided herein is a composition comprising an anti-BCMA antigen binding domain comprising a set of a variable light domain and a variable heavy domain selected from the following sets of variable light domains and variable heavy domains: a) a variable light domain having an amino acid of SEQ ID NO: 16 and a variable heavy domain having an amino acid of SEQ ID NO: 12; b) a variable light domain having an amino acid of SEQ ID NO: 26 and a variable heavy domain having an amino acid of SEQ ID NO: 22; c) a variable light domain having an amino acid of SEQ ID NO: 36 and a variable heavy domain having an amino acid of SEQ ID NO: 32; d) a variable light domain having an amino acid of SEQ ID NO: 46 and a variable heavy domain having an amino acid of SEQ ID NO: 42; e) a variable light domain having an amino acid of SEQ ID NO: 56 and a variable heavy domain having an amino acid of SEQ ID NO: 52; f) a variable light domain having an amino acid of SEQ ID NO: 66 and a variable heavy domain having an amino acid of SEQ ID NO: 62; and g) a variable light domain having an amino acid of SEQ ID NO: 76 and a variable heavy domain having an amino acid of SEQ ID NO: 72.

In another aspect, provided herein is a nucleic acid composition comprising: a) a first nucleic acid encoding a variable heavy domain of any of the BCMA ABD compositions provided herein; and b) a second nucleic acid encoding a variable light domain of any of the BCMA ABD compositions provided herein.

In another aspect, provided herein is an expression vector composition comprising: a) a first expression vector comprising the first nucleic acid; and b) a second expression vector comprising the second nucleic acid.

In one aspect, provided herein is a host cell comprising the expression vector composition. In yet another aspect, provided herein is a method of making a BCMA binding domain comprising culturing the host cell under conditions wherein said BCMA binding domain is expressed and recovering said BCMA binding domain.

### BRIEF DESCRIPTION OF THE DRAWINGS

FIG. 1 depicts the sequences for A) human, B) mouse, C) rat and D) cynomolgus BCMA. Underlined regions are the extracellular domain.

FIG. 2 depicts the sequence alignment of the extracellular domain of human BCMA with A) cynomolgus BCMA, B) mouse BCMA, and C) rat BCMA.

FIG. 3 depicts sequences for constant heavy domains that find use in embodiments of BCMA binding domain compositions described herein.

FIG. 4 depicts the constant domain of the cognate light chains which find use in the BCMA binding domain compositions described herein.

FIG. 5 depicts the variable heavy and variable light chain sequences for S1R4_10corr, an exemplary phage-derived BCMA binding domain, as well as the sequences for XENP23357, an anti-BCMA mAb based on SIR4_10corr and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems.

FIG. 6 depicts the variable heavy and variable light chain sequences for SIR5_125, an exemplary phage-derived BCMA binding domain, as well as the sequences for XENPXXX, an anti-BCMA mAb based on S1R5_125r and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems.

FIG. 7 depicts A) reduced LabChip gel, B) non-reduced LabChip gel, and C) analytical size-exclusion chromatogram of XENP23357 (BCMA mAb based on S1R4_10corr).

FIG. 8 depicts dissociation constant (K_{D}), association rate (Kₐ), and dissociation rate (K_{d}) for human and cynomolgus BCMA of phage-derived BCMA clones SIR5_125 and SIR4_10corr in the context of CD3 bsAb in a 1+1 format having monovalent binding for BCMA.

FIG. 9 depicts binding to A) human BCMA transfected 300-19 cells, B) cynomolgus BCMA transfected 300-19 cells, and C) parental 300-19 cells by phage-derived BCMA clones SIR5_125 and SIR4_10corr in the context of CD3 bsAbs in 1+1 and 2+1 formats respectively having monovalent and bivalent binding for BCMA.

FIG. 10 depicts induction of redirected T cell cytotoxicity by A) phage-derived BCMA clone SIR4_10corr and B) phage-derived BCMA clone SIR5_125 in the context of CD3 bsAbs in 1+1 and 2+1 formats respectively having monovalent and bivalent binding for BCMA. It should be noted that the 2+1 format utilizes a weaker affinity CD3 binding domain.

FIG. 11 depicts the variable heavy and variable light chain sequences for 1A1, an exemplary humanized hybridoma-derived BCMA binding domain, as well as the sequences for XENP24495, an anti-BCMA mAb based on 1A1 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems.

FIG. 12 depicts the variable heavy and variable light chain sequences for 1B4, an exemplary humanized hybridoma-derived BCMA binding domain, as well as the sequences for XENP24503, an anti-BCMA mAb based on 1B4 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems.

FIG. 13 depicts the variable heavy and variable light chain sequences for 1B3, an exemplary humanized hybridoma-derived BCMA binding domain, as well as the sequences for XENP24617, an anti-BCMA mAb based on 1B3 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems.

FIG. 14 depicts the variable heavy and variable light chain sequences for 5F2, an exemplary humanized hybridoma-derived BCMA binding domain, as well as the sequences for XENP24621, an anti-BCMA mAb based on 5F2 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems.

FIG. 15 depicts the variable heavy and variable light chain sequences for 6E3, an exemplary humanized hybridoma-derived BCMA binding domain, as well as the sequences for XENP24625, an anti-BCMA mAb based on 6E3 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems.

FIG. 16 depicts A) reduced LabChip gel and B) non-reduced LabChip gel for XENP24495 (1A1), XENP24503 (1B4), XENP24617 (1B3), XENP24621 (5F2), and XENP24625 (6E3).

FIG. 17 depicts analytical size-exclusion chromatograms for A) XENP24495 (1A1), B) XENP24503 (1B4), C) XENP24617 (1B3), D) XENP24621 (5F2), and E) XENP24625 (6E3)

FIG. 18 depicts dissociation constant (K_{D}), association rate (Kₐ), and dissociation rate (Kₐ) for human and cynomolgus BCMA of humanized hybridoma-derived BCMA clones 1B4, 1B3, 5F2, 6E3, and 1A1 in the context of CD3 bsAb in a 1+1 format having monovalent binding for BCMA.

FIG. 19 depicts binding to A) human BCMA transfected 300-19 cells, B) cynomolgus BCMA transfected 300-19 cells, and C) parental 300-19 cells by humanized hybridoma-derived BCMA clones S1R4_10corr, 1B4, 1B3, 5F2, 6E3, and 1A1 in the context of bivalent monospecific mAbs.

FIG. 20 depicts induction of redirected T cell cytotoxicity by humanized hybridoma-derived BCMA clones 1A1, 1B4, 1B3, 5F2, and 6E3 in the context of CD3 bsAbs in 1+1 and 2+1 formats respectively having monovalent and bivalent binding for BCMA. It should be noted that the 2+1 bsAbs utilize lower affinity CD3 binding domain.

FIG. 21 depicts pharmacokinetics of humanized hybridoma-derived BCMA clones 1A1, 1B4, 1B3, 5F2, and 6E3 in the context of CD3 bsAbs in 1+1 having monovalent binding for BCMA.

FIG. 22 depicts exemplary formats of the anti-BCMA heterodimeric antibodies provided herein. FIG. 22A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 22B depicts the “2+1 Fab₂-scFv-Fc” format, with a first Fab arm and a second Fab-scFv arm, wherein the Fab binds a first antigen and the scFv binds second antigen. The 2+1 Fab₂-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 22C depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen.

FIG. 23A-33F depict useful pairs of heterodimerization variant sets (including skew and pI variants) that can be used in the anti-BCMA heterodimeric antibodies provided herein. In FIG. 23F, there are variants for which there are no corresponding “monomer 2” variants. Such variants are pI variants that can be used alone on either monomer of an anti-BCMA heterodimeric antibody, or included, for example, on the non-scFv side of a format that utilizes an scFv as a component and an appropriate charged scFv linker can be used on the second monomer that utilizes an scFv. Suitable charged linkers are shown in FIG. 26.

FIG. 24 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 25 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 26 depicts a number of charged scFv linkers that find use in increasing or decreasing the pI of the subject heterodimeric anti-BCMA heterodimeric antibodies that utilize one or more scFv as a component, as described herein. A single prior art scFv linker with a single charge is referenced as “Whitlow”, from Whitlow et al., Protein Engineering 6 (8): 989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab₂-scFv-Fc formats).

FIG. 27 depicts a number of exemplary domain linkers. In some embodiments, these linkers find use linking a single-chain Fv to an Fc chain. In some embodiments, these linkers may be combined in any orientation. For example, a GGGGS linker may be combined with a “lower half hinge” linker at the N-terminus or at the C-terminus. In some embodiments, two or more of the domain linkers depicted in FIG. 27 can be combined to form longer domain linkers for use in the heterodimeric antibodies described herein.

FIG. 28 shows a particularly useful embodiment of the heterodimeric Fc domains (i.e. CH2-CH3 in this embodiment) of the the anti-BCMA heterodimeric antibodies the anti-BCMA heterodimeric antibodies of the invention.

FIG. 29 depicts various heterodimeric skewing variant amino acid substitutions that can be used with the heterodimeric antibodies described herein.

FIGS. 30A-30C show the sequences of several useful the anti-BCMA heterodimeric antibody backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats. Additionally, C-terminal glycine and/or lysine deletion may occur naturally for example during production and storage.

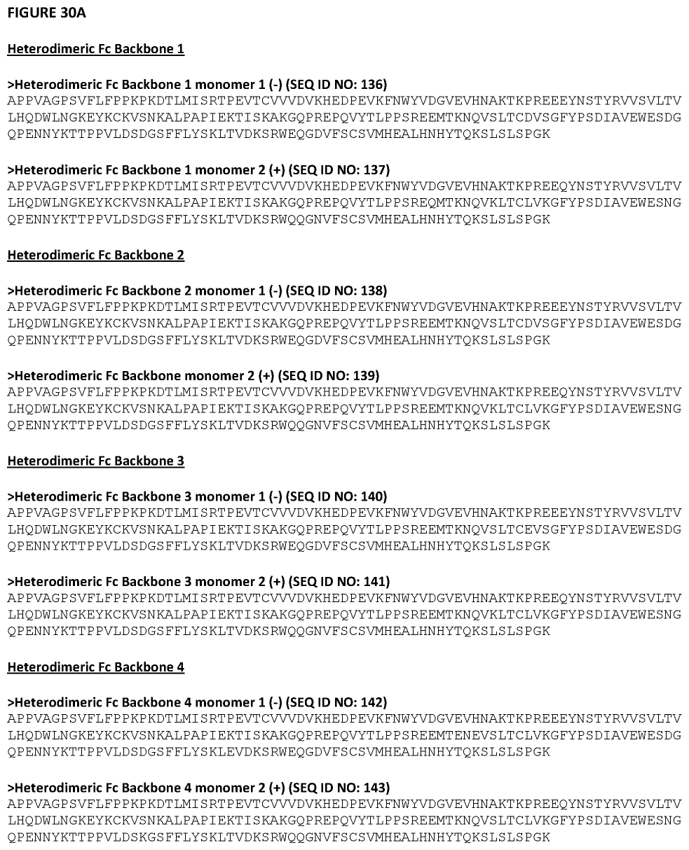

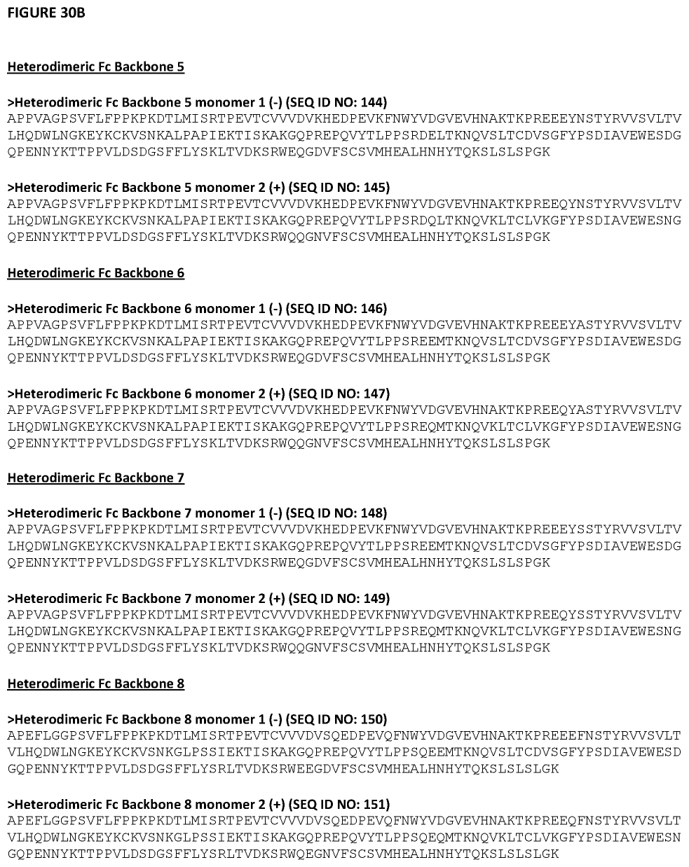

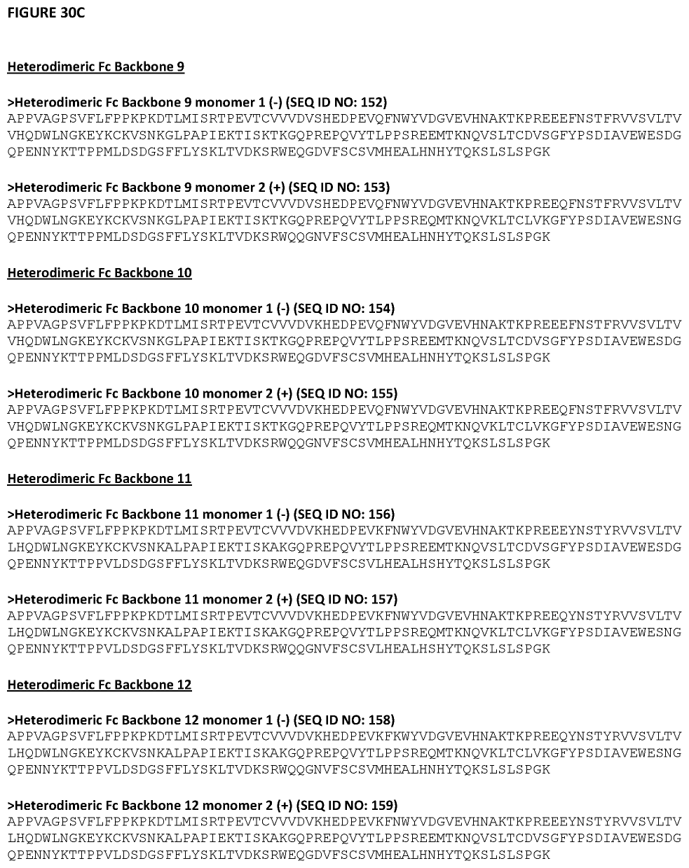

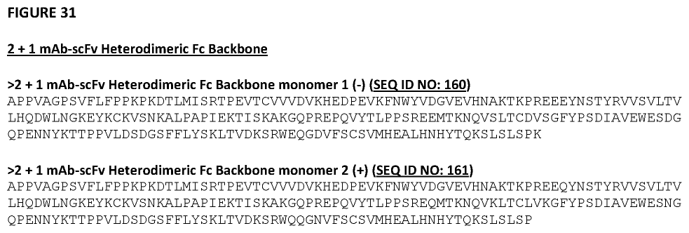

FIG. 31 depicts illustrative sequences of heterodimeric the anti-BCMA heterodimeric antibodies for use in the 2+1 mAb-scFv format. The format depicted here is based on heterodimeric Fc backbone 1 as depicted in FIG. X, except further including G446_ on monomer 1 (−) and G446_/K447_ on monomer 2 (+). It should be noted that any of the additional backbones depicted in FIG. 30 may be adapted for use in the 2+1 mAb-scFv format with or without including K447_ on one or both chains. It should be noted that these sequences may further include the M428L/N434S variants.

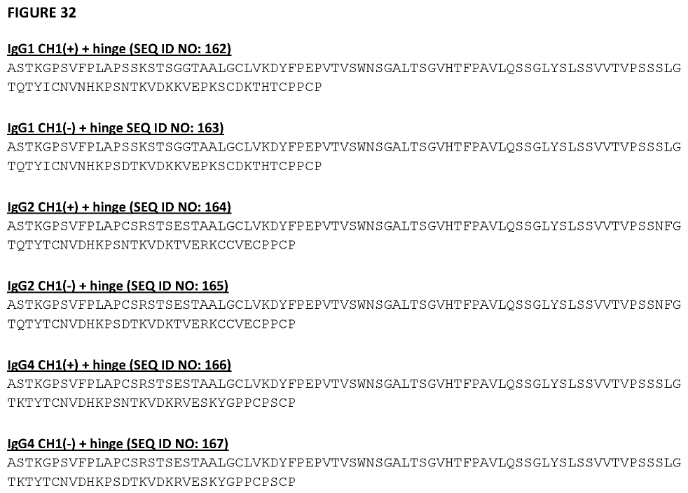

FIG. 32 depicts sequences for “CH1+hinge” that find use in embodiments of the anti-BCMA antibodies that utilize a Fab a binding domain. The “CH1+hinge” sequences find use linking the variable heavy domain (V_{H}) to the Fc backbones. For particular embodiments wherein the Fab is on the (+) side, the “CH1(+)+hinge” sequences may find use. For particular embodiments wherein the Fab is on the (−) side, the “CH1(−)+hinge” sequences may find use.

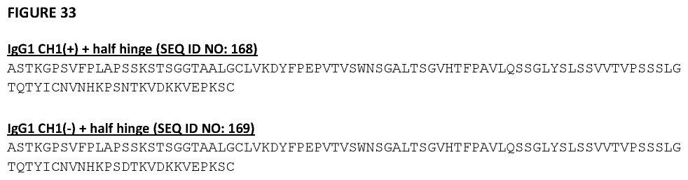

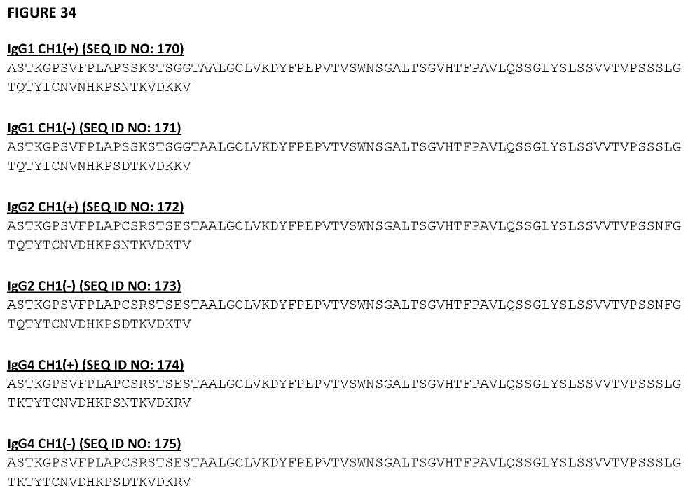

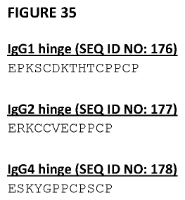

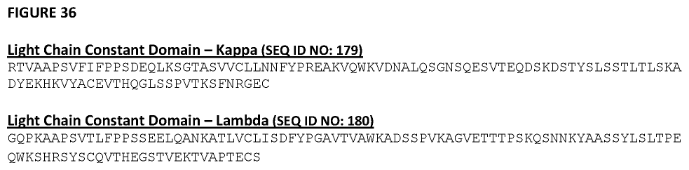

FIG. 33 depicts sequences for “CH1+half hinge” domain linker that find use in embodiments of the anti-BCMA heterodimeric antibodies in the 2+1 Fab₂-scFv-Fc. In the 2+1 Fab₂-scFv-Fc format, the “CH1+half hinge” sequences find use linking the variable heavy domain (V_{H}) to the scFv domain on the Fab-scFv-Fc side of the bispecific antibody. It should be noted that other linkers may be used in place of the “CH1+half hinge”. It should also be noted that although the sequences here are based on the IgG1 sequence, equivalents can be constructed based on the IgG2 or IgG4 sequences.

FIG. 34 depicts sequences for “CH1” that find use in embodiments of the anti-BCMA heterodimeric antibodies.

FIG. 35 depicts sequences for “hinge” that find use in embodiments of the anti-BCMA heterodimeric antibodies.

FIG. 36 depicts the constant domain of the cognate light chains which find use in the antibodies depicted herein.

### DETAILED DESCRIPTION

### I. Overview

Provided herein are novel BCMA antigen binding domain compositions and antibodies that include such BCMA antigen binding domains. In some embodiments, the anti-BCMA antibodies are heterodimeric and bispecific antibodies that include at least one of the BCMA antigen binding domains provided herein. In exemplary embodiments, the BCMA antibody does not include a CD3 binding domain. The BCMA antigen binding domains and antibodies provided herein are useful, for example, in the treatment of BCMA-associated diseases.

### II. Definitions

In order that the application may be more completely understood, several definitions are set forth below. Such definitions are meant to encompass grammatical equivalents.

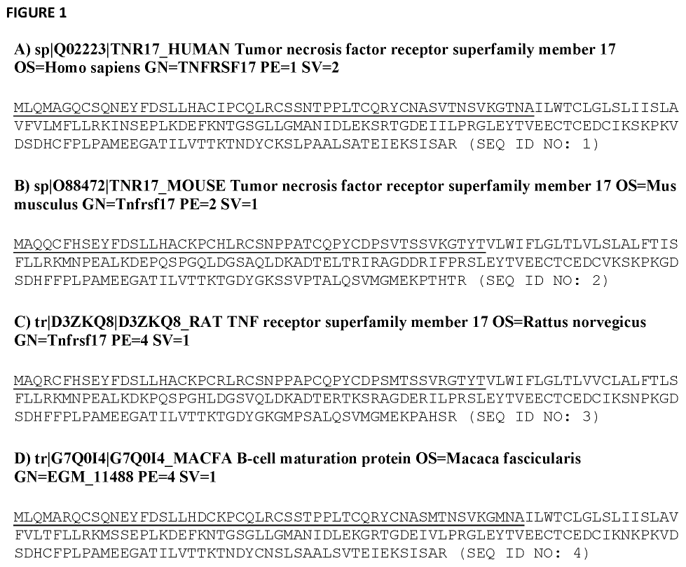

By “B-cell maturation antigen” or “BCMA” or “tumor necrosis factor receptor superfamily member 17” or TNFRSF17 or CD269 herein is meant a single-pass type III transmembrane glycoprotein in the TNF-receptor superfamily that is primarily expressed on the surface of plasma cells and is encoded by the TNFRSF17 gene in humans (e.g., Genebank Accession Numbers NM_001192 and NP_001183 (human); and NM_0011608 and NP_035738 (mouse)). BCMA binds to APRIL (TNFSF13) and BAFF (TNFSF13B), members of the TNF ligand superfamily. BCMA is expressed in immune organs and mature B cells (plasma cells), as well as cancer cells such as glioblastoma, multiple myeloma, chronic lympcytic leukemia and Hodgkin lymphoma. Deshayes et al., Oncogene 23 (17): 3005-3012 (2004); Novak et al., Blood 103 (2): 689-694 (2004); Novak et al., Blood 100 (8): 2973-2979 (2002); Chiu et al., Blood 109 (2): 729-739 (2007). Exemplary BCMA sequences are depicted in FIG. 1.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

As used herein, the term “antibody” refers to traditional immunoglobulin (Ig) antibodies unless stated specifically otherwise.

Traditional immunoglobulin (Ig) antibodies are “Y” shaped tetramers. Each tetramer is typically composed of two identical pairs of polypeptide chains, each pair having one “light chain” monomer and one “heavy chain” monomer.

An antibody heavy chain typically includes a variable heavy (VH) domain (also referred to as “a heavy chain variable domain”), which includes vhCDR1-3, and an Fc domain, which includes a CH2-CH3 monomer. In some embodiments, an antibody heavy chain includes a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). In some embodiments, the Fc domain is from IgG1, IgG2, IgG3 or IgG4, with IgG1 hinge-CH2-CH3 and IgG4 hinge-CH2-CH3 finding particular use in many embodiments. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” or “constant heavy domain” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index as in Kabat.

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference. Other numbering conventions are available in the art and those skilled in the art would readily be able to determine the exact numbering and placement in those other numbering convention systems based on what's described herein.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL) (also referred to as “light chain variable domain”), which includes light chain CDRs vlCDR1-3, and a constant light chain region or light chain constant region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of antibody sequences, specifically binds a target antigen (e.g., BCMA) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 variable light CDRs. The CDRs are present in the variable heavy domain (vhCDR1-3) and variable light domain (vlCDR1-3). The variable heavy domain and variable light domain form an Fv region.

Typically, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, i.e., a heavy and light chain respectively.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27 (1): 55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)). Those skilled in the art would readily be able to determine the exact numbering and placement of the CDRs in other numbering systems described herein and known in the art.

The CDRs contribute to the formation of the antigen-binding, or more specifically, epitope binding site of the antibody. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The six CDRs of the subject antibodies are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain).

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution M428L refers to a variant polypeptide, in this case an Fc variant, in which the methionine at position 272 is replaced with leucine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below.

As described below, in some embodiments the parent polypeptide, for example an Fc parent polypeptide, is a human wild type sequence, such as the heavy constant domain or Fc region from IgG1 or IgG2, although human sequences with variants can also serve as “parent polypeptides”, for example the IgG1/2 hybrid of US Publication 2006/0134105 can be included. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1 or IgG2.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). Those skilled in the art would readily be able to determine the corresponding positions in other numbering systems described herein and known in the art. The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides, and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “non-naturally occurring modification” as used herein is meant an amino acid modification that is not isotypic. For example, because none of the known human IgGs comprise a serine at position 434, the substitution 434S in IgG1 or IgG2 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody that is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction using an assay described herein or known in the art. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope. A suitable control molecule is described herein including the Example sections and known in the art.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### III. BCMA Binding Domains

In one aspect, provided herein are BCMA antigen binding domains (ABDs) that bind BCMA, and antibodies that include such BCMA antigen binding domains (e.g., the heterodimeric antibodies provided herein). The BCMA ABDs provided herein generally include a variable heavy domain (VH) and a variable light domain (VL). In exemplary embodiments, the BCMA is capable of binding to human BCMA (see FIG. 1).

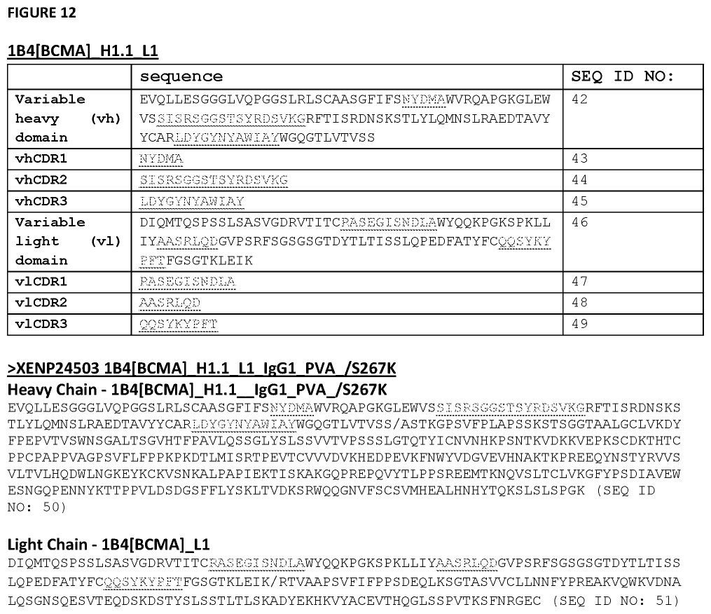

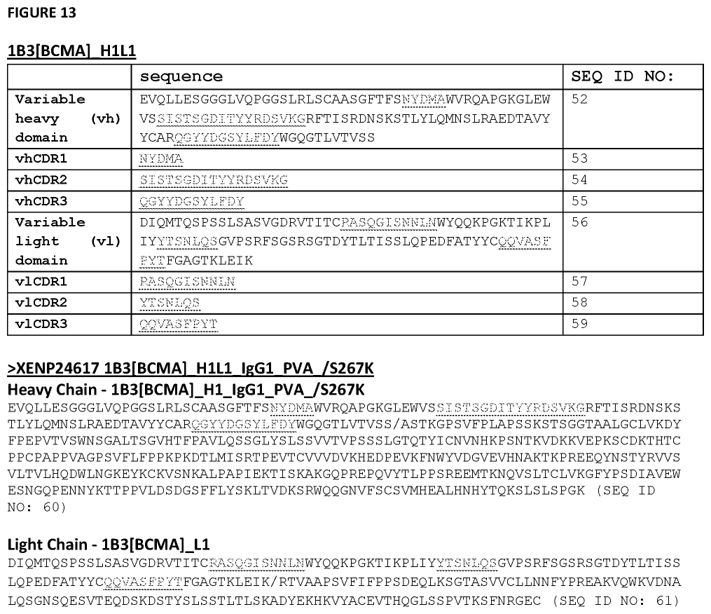

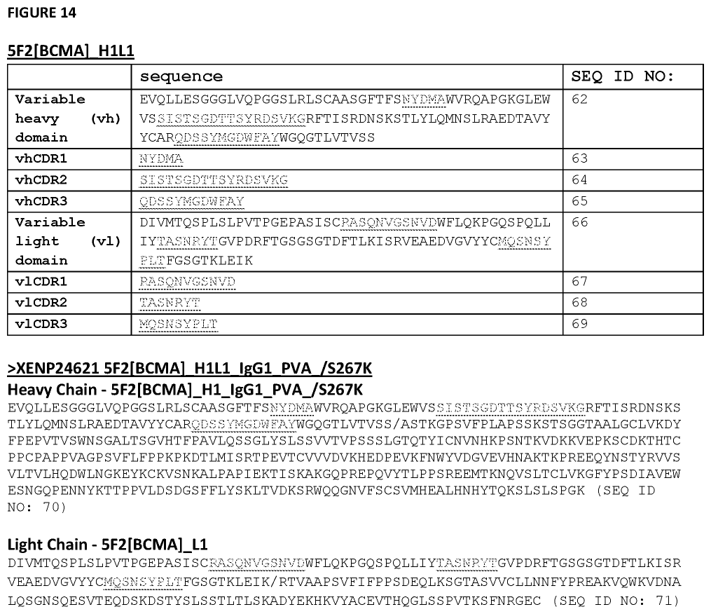

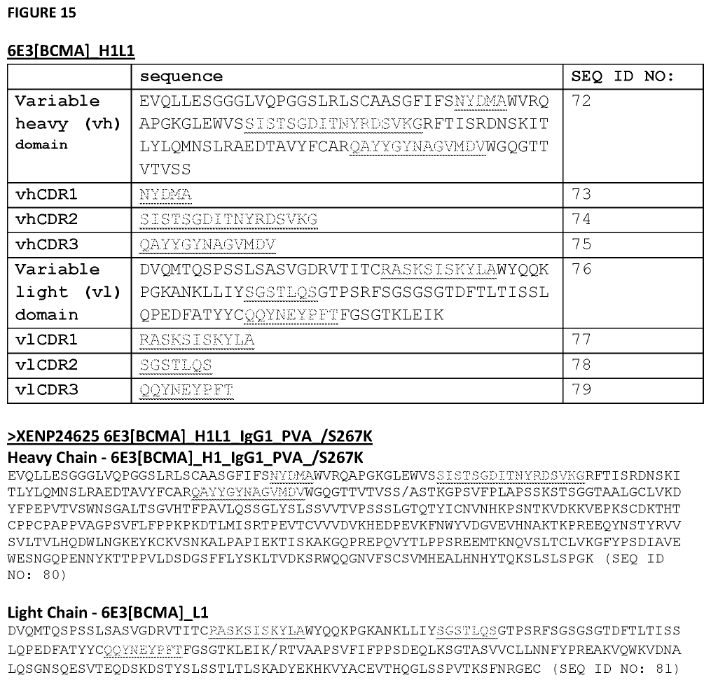

As will be appreciated by those in the art, suitable BCMA binding domains can comprise a set of 6 CDRs as depicted in the figures, either as they are underlined or, in the case where a different numbering scheme is used as described herein and as shown in Table 2, as the CDRs that are identified using other alignments within the variable heavy (VH) domain and variable light domain (VL) sequences of those depicted in FIGS. 5, 6 and 11-15 and the Sequence Listing. Suitable BCMA ABDs can also include the entire VH and VL sequences as depicted in these sequences and figures, used as scFvs or as Fabs.

In one embodiment, the BCMA antigen binding domain includes the 6 CDRs (i.e., vhCDR1-3 and vlCDR1-3) of any of the BCMA binding domains described herein, including the figures. In some embodiments, the BCMA ABD is one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15).

In addition to the parental CDR sets disclosed in the figures that form the BCMA ABDs (e.g., FIGS. 5, 6 and 11-15), provided herein are variant BCMA ABDS having CDRs that include at least one modification of the BCMA ABD CDRs disclosed herein (e.g., FIGS. 5, 6 and 11-15). In one embodiment, the BCMA ABD includes a set of 6 CDRs with 1, 2, 3, 4, 5, 6, 7, 8, 9, 10 amino acid modifications as compared to the 6 CDRs of a BCMA ABD as described herein, including the figures. In exemplary embodiments, the BCMA ABD includes a set of 6 CDRs with 1, 2, 3, 4, 5, 6, 7, 8, 9, 10 amino acid modifications as compared to the 6 CDRs of one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15). In certain embodiments, the BCMA ABD is capable of binding BCMA antigen, as measured by at least one of a Biacore, surface plasmon resonance (SPR) and/or BLI (biolayer interferometry, e.g., Octet assay) assay, with the latter finding particular use in many embodiments. In particular embodiments, the BCMA ABD is capable of binding human BCMA antigen (see FIG. 1).

In some embodiments, the BCMA ABD includes 6 CDRs that are at least 90, 95, 97, 98 or 99% identical to the 6 CDRs of a BCMA ABD as described herein, including the figures. In exemplary embodiments, the BCMA ABD includes 6 CDRs that are at least 90, 95, 97, 98 or 99% identical to the 6 CDRs of one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15). In certain embodiments, the BCMA ABD is capable of binding BCMA antigen, as measured by at least one of a Biacore, surface plasmon resonance (SPR) and/or BLI (biolayer interferometry, e.g., Octet assay) assay, with the latter finding particular use in many embodiments. In particular embodiments, the BCMA ABD is capable of binding human BCMA antigen (see FIG. 1).

In another exemplary embodiment, the BCMA ABD include the variable heavy (VH) domain and variable light (VL) domain of any one of the BCMA ABDs described herein, including the figures. In exemplary embodiments, the BCMA ABD is one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15).

In addition to the parental BCMA variable heavy and variable light domains disclosed herein, provided herein are BCMA ABDs that include a variable heavy domain and/or a variable light domain that are variants of a BCMA ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a BCMA ABD described herein, including the figures. In some embodiments, the variant VH domain and/or VL domain has includes 1, 2, 3, 4, 5, 6, 7, 8, 9 or 10 amino acid changes in a framework region (e.g., FR1, FR2, FR3, or FR4) from a VH and/or VL domain of a BCMA ABD described herein, including the figures. In exemplary embodiments, the variant VH domain and/or VL domain has from 1, 2, 3, 4, 5, 6, 7, 8, 9 or 10 amino acid changes from a VH and/or VL domain of one of the following BCMA ABDs: SIR4_10corr[BCMA]_HIL1, SIR5_125 [BCMA]_HIL1, 1A1 [BCMA]_HIL1, 1B4 [BCMA] _H1L1, 1B3 [BCMA]_HIL1, 5F2 [BCMA]_HIL1, and 6E3 [BCMA]_HIL1 (FIGS. 5, 6, and 11-15). In exemplary embodiments, the variant VH domain and/or VL domain has from 1, 2, 3, 4, 5, 6, 7, 8, 9 or 10 amino acid changes in a framework region (e.g., FR1, FR2, FR3, or FR4) of the VH and/or VL domain of one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15). In certain embodiments, the BCMA ABD is capable of binding to BCMA, as measured at least one of a Biacore, surface plasmon resonance (SPR) and/or BLI (biolayer interferometry, e.g., Octet assay) assay, with the latter finding particular use in many embodiments. In particular embodiments, the BCMA ABD is capable of binding human BCMA antigen (see FIG. 1).

In one embodiment, the variant VH and/or VL domain is at least 90, 95, 97, 98 or 99% identical to the VH and/or VL of a BCMA ABD as described herein, including the figures. In exemplary embodiments, the variant VH and/or VL domain is at least 90, 95, 97, 98 or 99% identical to the VH and/or VL of one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15). In certain embodiments, the BCMA ABD is capable of binding to BCMA, as measured by at least one of a Biacore, surface plasmon resonance (SPR) and/or BLI (biolayer interferometry, e.g., Octet assay) assay, with the latter finding particular use in many embodiments. In particular embodiments, the BCMA ABD is capable of binding human BCMA antigen (see FIG. 1).

### IV. Antibodies

In one aspect, provided herein are anti-BCMA antibodies that include any of the BCMA antigen binding domains described herein. In some embodiments, the anti-BCMA antibody includes 1, 2, 3, 4, 5, 6, 7, 8, 9 or 10 of the BCMA ABDs described herein. In some embodiments, the anti-BCMA antibody is a monospecific bivalent antibody. In some embodiments, the anti-BCMA antibody is a multi-specific antibody that includes at least one BCMA ABDs described herein. In some embodiments, the multi-specific antibody binds at least 2, 3, 4, or 5 different antigens. In some embodiments, the multi-specific antibody does not bind CD3 antigen. In some embodiments, the antibody is a bispecific antibody that includes a BCMA ABD provided herein. In certain embodiments, the antibody is a “1+1 Fab-scFv-Fc” or “2+1 Fab₂-scFv-Fc” bispecific format antibody and includes at least one of the BCMA binding domains provided herein.

The antibodies provided herein include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

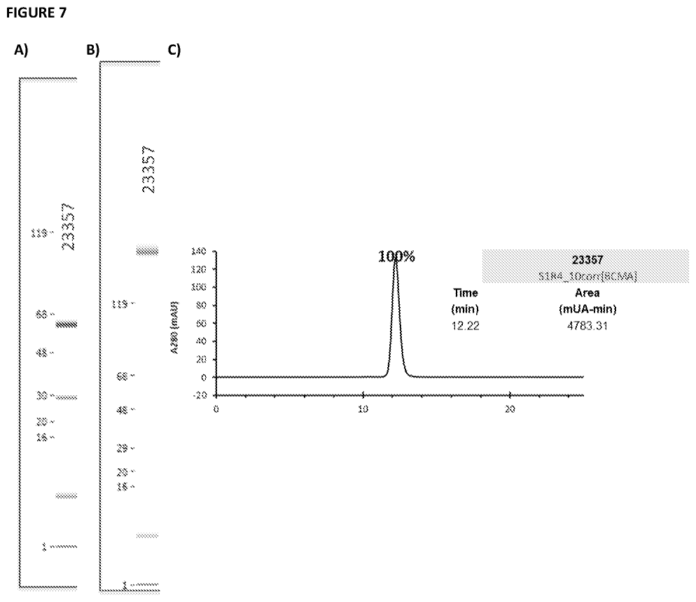

As shown herein, there are a number of suitable linkers (for use as either domain linkers or scFv linkers) that can be used to covalently attach the recited domains (e.g., scFvs, Fabs, Fc domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example, the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker”, used to link any two domains as outlined herein together. For example, in the 2+1 Fab₂-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used.

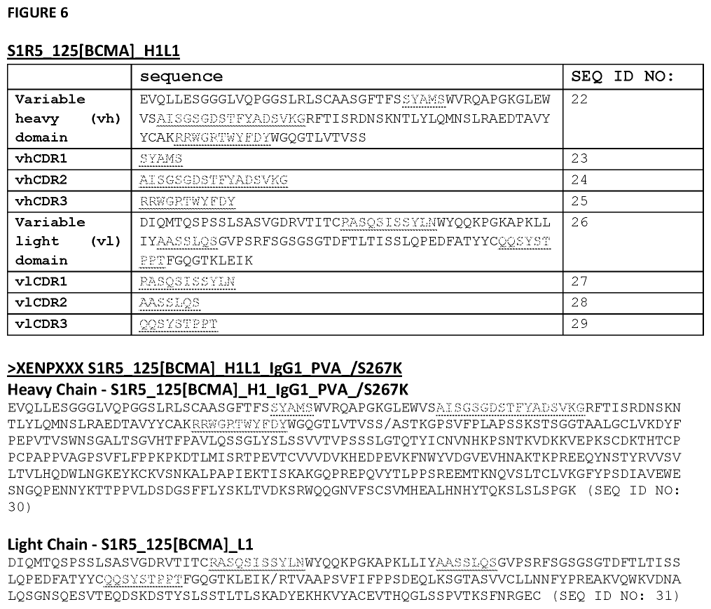

In some embodiments, the linker is a scFv linker that is used to covalently attach the VH and VL domains as discussed herein. In many cases, the scFv linker is a charged scFv linker, a number of which are shown in FIG. 6. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the invention as well, and thus those included in FIG. 6 can be used in any embodiment herein where a linker is utilized.

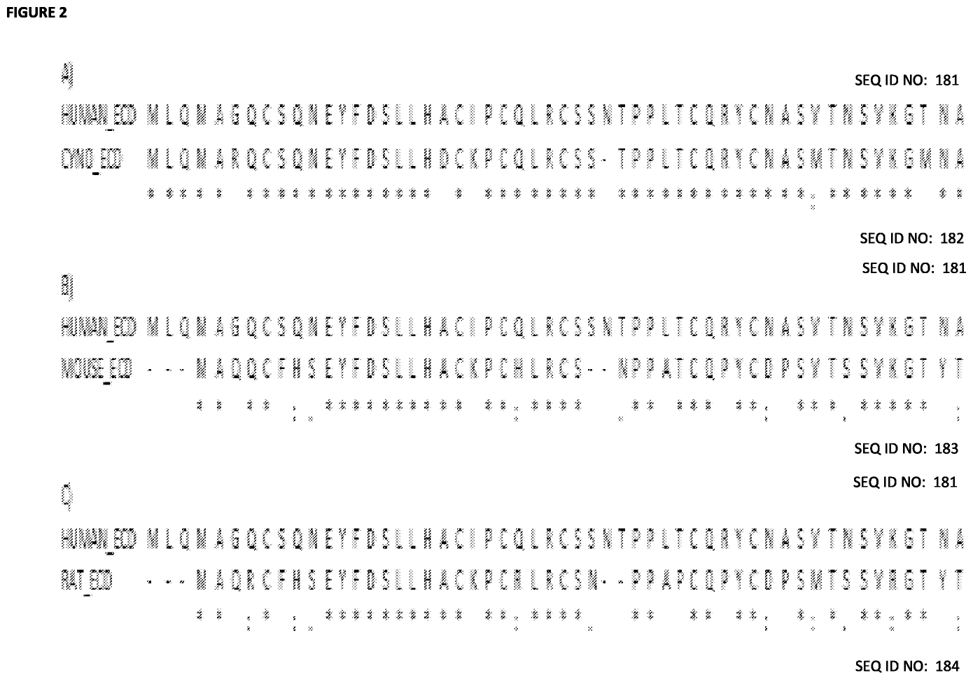

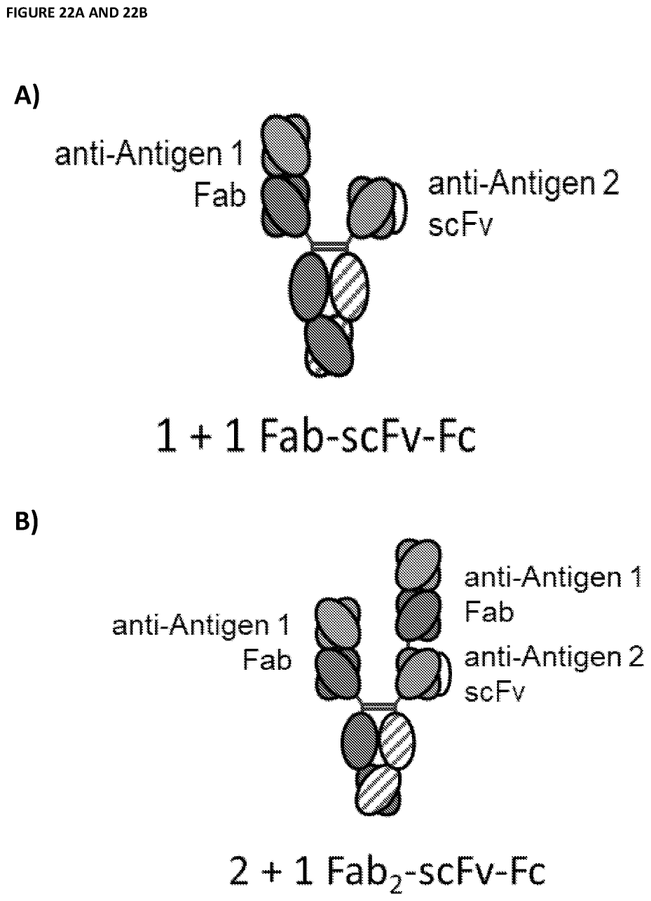

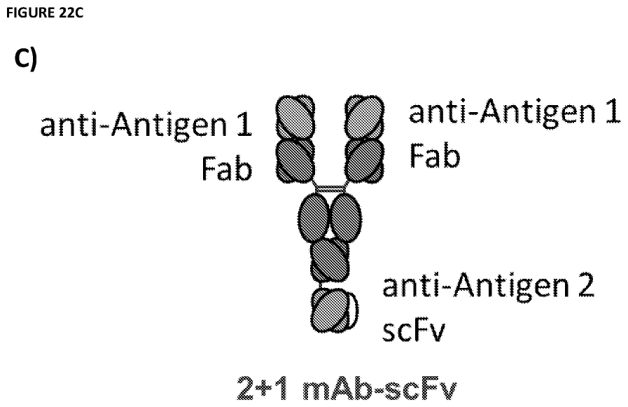

The BCMA antigen binding domains provided can be included in any useful antibody format including, for example, canonical immunoglobulin, as well as the “1+1 Fab-scFv-Fc,” and “2+1 Fab₂-scFv-Fc” formats provided herein (see, e.g., FIG. 22). Other useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in US20180127501A1, which is incorporated by reference herein, particularly in pertinent part relating to antibody formats (see, e.g., FIG. 2).

In some embodiments, the subject antibody includes one or more of the BCMA ABDs provided herein. In some embodiments, the antibody includes one BCMA ABD. In other embodiments, the antibody includes two BCMA ABDs. In exemplary embodiments, the BCMA ABD includes the variable heavy domain and variable light domain of one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15).

In an exemplary embodiment, the antibody is a bispecific antibody that includes one or two BCMA ABDs, including any of the BCMAABDs provided herein. Bispecific antibodies that include such BCMA ABDs include, for example, “1+1 Fab-scFv-Fc,” “2+1 Fab₂-scFv-Fc,” “bispecifics format antibodies (FIG. 22). In exemplary embodiments, the BCMA ABD is one of the following BCMA ABDs: S1R4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15). In exemplary embodiments, the bispecific antibody does not bind CD3.

### A. Heterodimeric Antibodies

In exemplary embodiments, the anti-BCMA antibodies provided herein are heterodimeric bispecific antibodies that include one of the BCMA antigen binding domains described herein. In exemplary embodiments, the heterodimeric antibody includes two variant Fc domain sequences. Such variant Fc domains include amino acid modifications to facilitate the self-assembly and/or purification of the heterodimeric antibodies. In certain embodiments, the heterodimeric antibody does not bind CD3.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

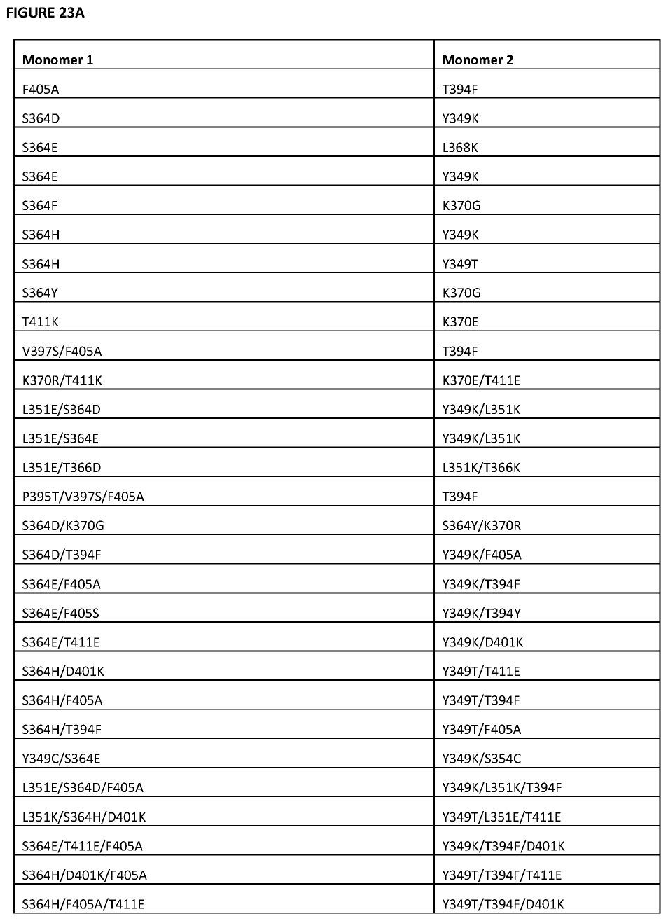

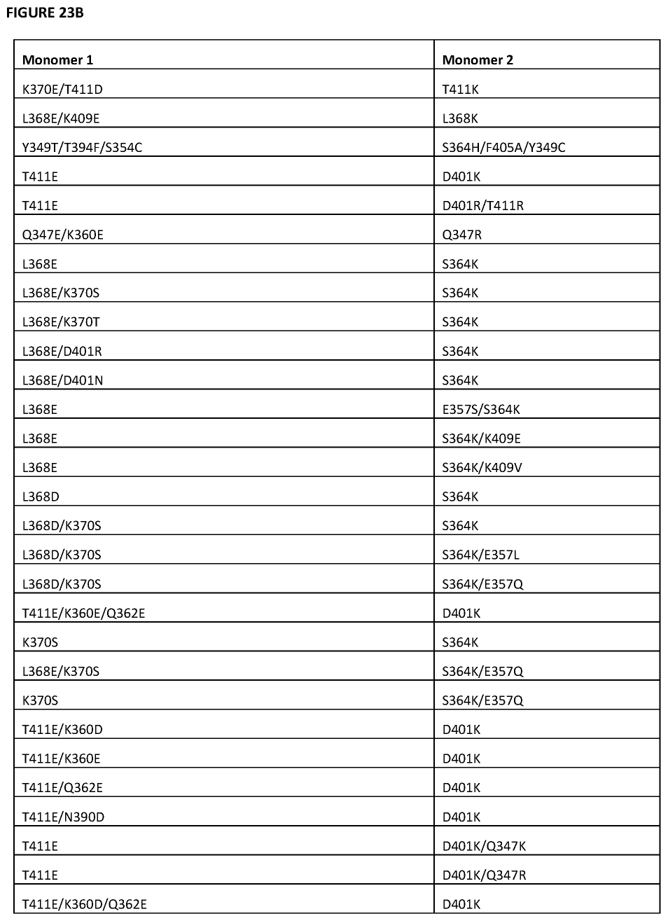

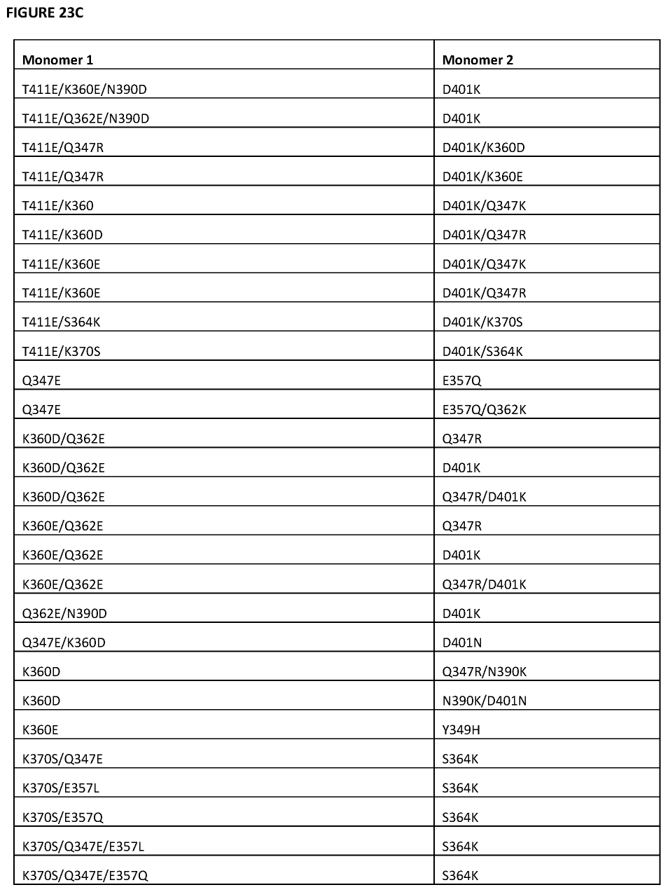

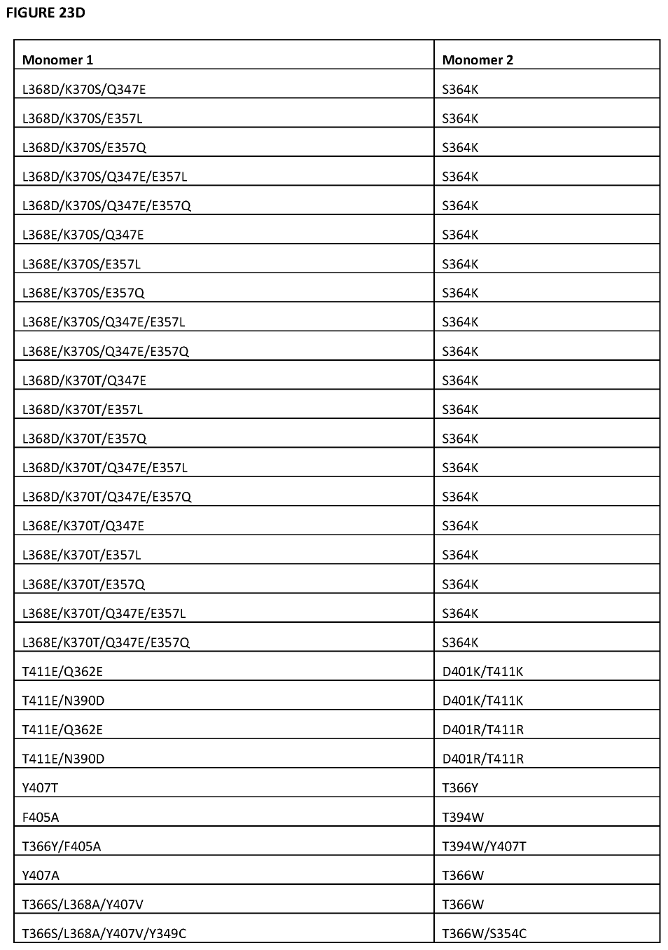

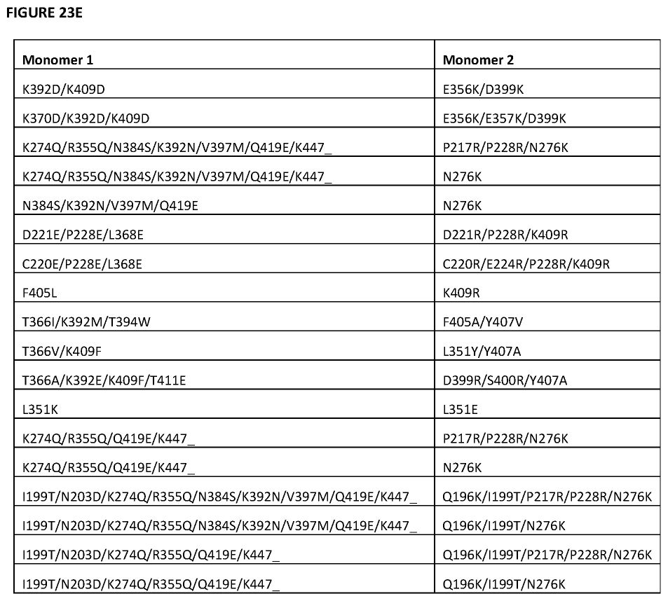

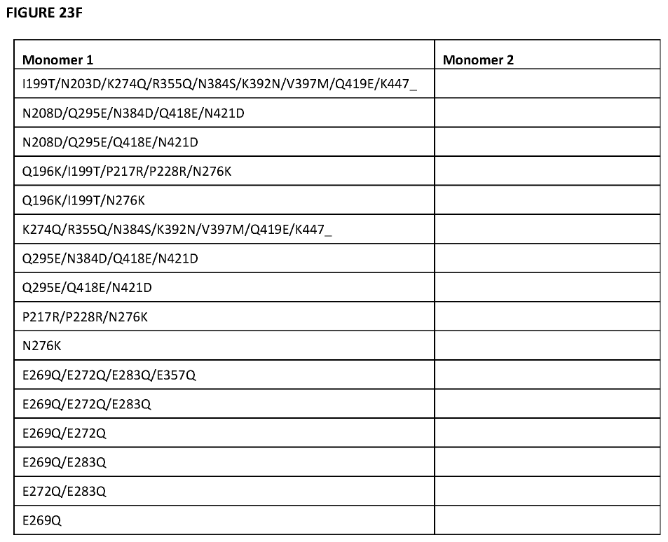

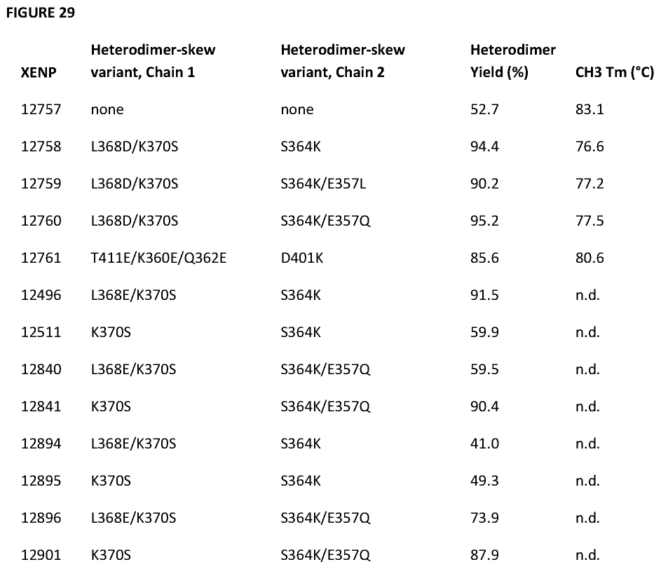

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 23 and 29.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9 (7): 617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285 (25): 19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A: 50% heterodimer A/B: 25% homodimer B/B). In exemplary embodiments, the heterodimeric antibody includes a S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C or T366S/L368A/Y407V/S354C:T366W/Y349C) “skew” variant amino acid substitution set. In an exemplary embodiment, the heterodimeric antibody includes a “S364K/E357Q:L368D/K370S” amino acid substitution set (EU numbering). In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

### 2. pI (Isoelectric Point) Variants for Heterodimers

In some embodiments, the heterodimeric antibody includes purification variants that advantageously allow for the separation of heterodimeric antibody from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format and the “2+1 Fab₂-scFv-Fc” format allow separation on the basis of size. As described above, it is also possible to “skew” the formation of heterodimers over homodimers using skew variants. Thus, a combination of heterodimerization skew variants and pI variants find particular use in the heterodimeric antibodies provided herein.

Additionally, as more fully outlined below, depending on the format of the heterodimeric antibody, pI variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab₂-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine).

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, and 2+1 Fab₂-scFv-Fc, formats, the starting pI of the scFv (1+1 Fab-scFv-Fc, 2+1 Fab₂-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g., Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize, and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

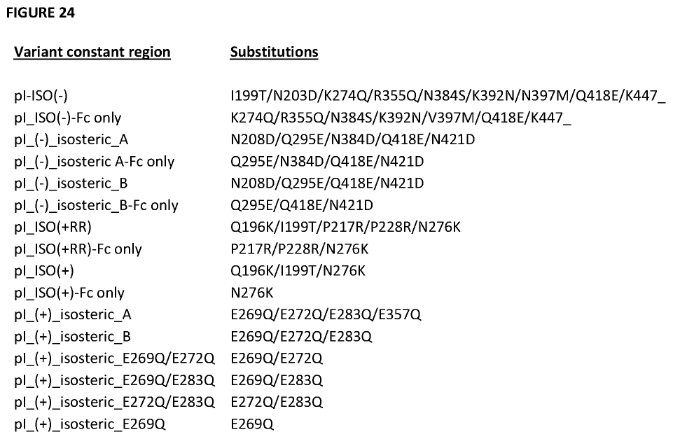

Exemplary combinations of pI variants are shown in FIGS. 24 and 25, and FIG. 30 of US Publ. App. No. 2016/0355608, all of which are herein incorporated by reference in its entirety and specifically for the disclosure of pI variants.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄. However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1). Additional pI variants that can be used in the heterodimeric antibodies provided herein are shown in FIGS. 23 and 24.

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an anti-BCMA antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

### 3. Isotypic Variants

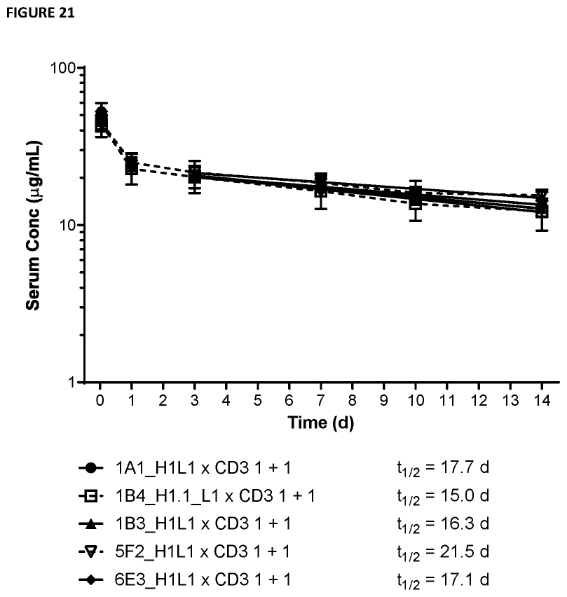

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

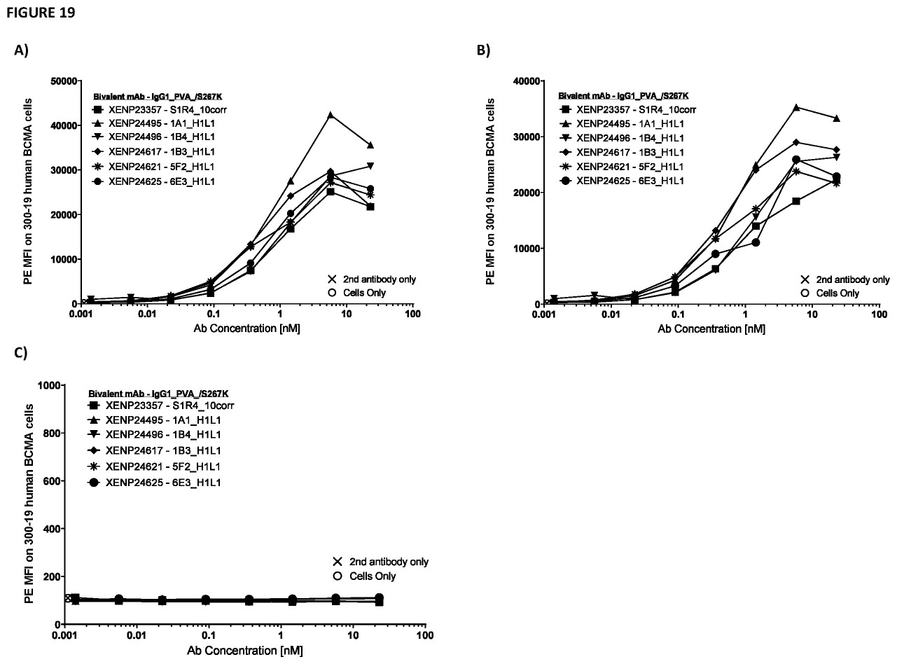

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18 (12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23 (5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### B. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T, according to EU numbering. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 436I or V/434S, 436V/428L and 259I/308F/428L. Such modification may be included in one or both Fc domains of the subject antibody. In exemplary embodiments, the antibody includes 428L/434S modifications (EU numbering).

### 2. Ablation Variants

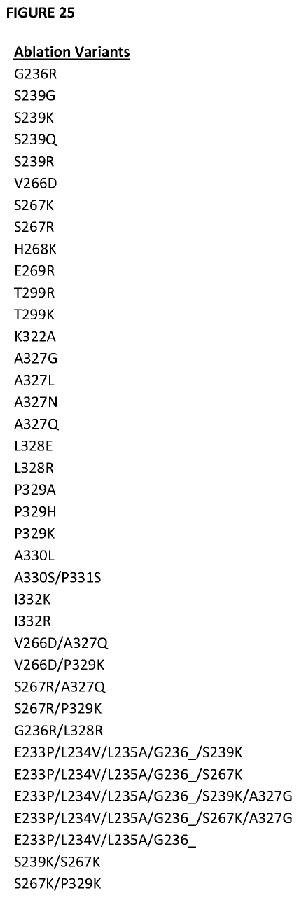

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind BCMA monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del (EU numbering). It should be noted that the ablation variants referenced herein ablate FcγR binding but generally not FcRn binding. Additional ablation variants that can be included in the heterodimeric antibodies provided herein are show in FIGS. 24 and 25.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### C. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants, skew, and pI, are also independently and optionally combined with Fc ablation variants, Fc variants, FcRn variants, as generally outlined herein.

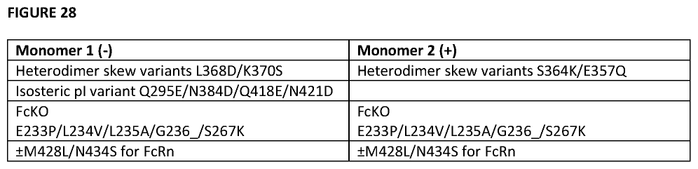

Exemplary combination of variants that are included in some embodiments of the heterodimeric 1+1 Fab-scFv-Fc, and 2+1 Fab₂-scFv-Fc format antibodies are included in FIG. 28. In some embodiments, the heterodimeric antibody includes a combination of variants as depicted in FIG. 28. In certain embodiments, the antibody is a heterodimeric 1+1 Fab-scFv-Fc, or 2+1 Fab₂-scFv-Fc, format antibody. In exemplary embodiments, the antibody includes at least one of the BCMA ABDs provided herein and does not bind CD3.

### D. Useful Antibody Formats

As will be appreciated by those in the art and discussed more fully below, the heterodimeric bispecific antibodies provided herein can take on several different configurations as generally depicted in FIG. 22.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain.

The present invention utilizes at least one BCMA antigen binding domain. As will be appreciated by those in the art, any collection of CDRs, variable light and variable heavy domains, Fabs and scFvs as depicted in any of the figures (see particularly FIGS. 5, 6, and 11-15) and variants thereof can be used.

### 1. 1+1 Fab-scFv-Fc Format (“Bottle Opener”)

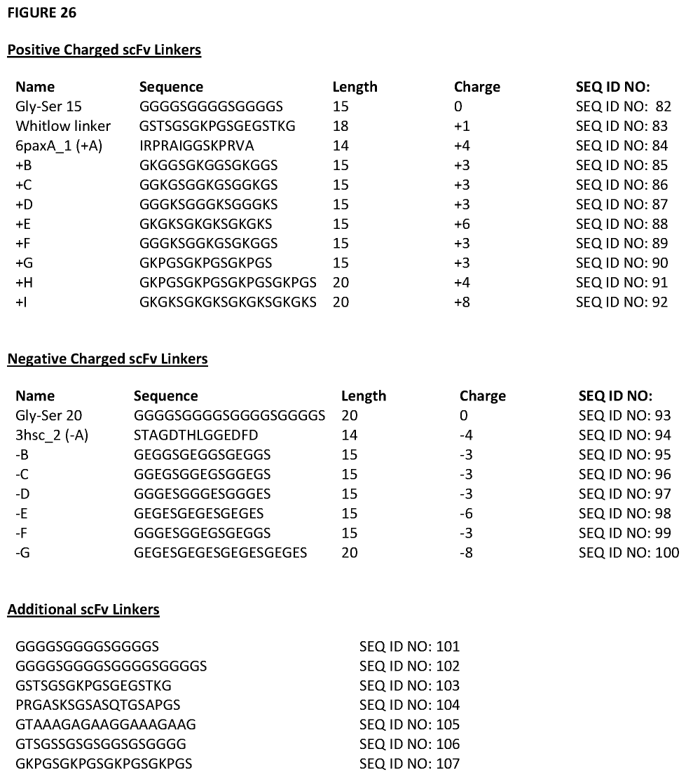

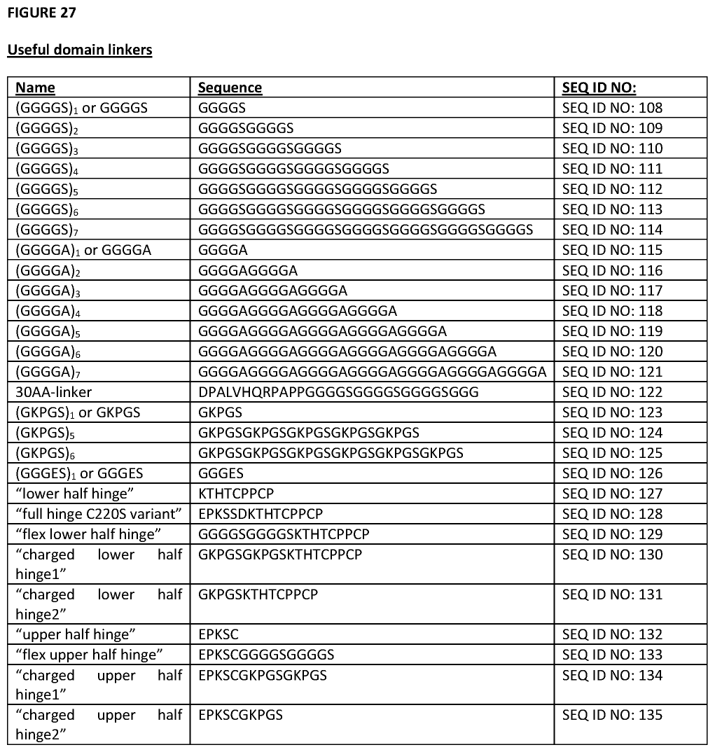

One heterodimeric antibody format that finds particular use in subject bispecific antibodies provided herein is the “1+1 Fab-scFv-Fc” or “bottle opener” format as shown in FIG. 22A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv”, as defined below) and a second Fc domain. The scFv includes a second variable heavy domain (VH2) and a second variable light domain (VL2), wherein the VH2 is attached to the VL2 using an scFv linker that can be charged (see, e.g., FIG. 26). The scFv is attached to a second Fc domain using a domain linker (see, e.g., FIG. 27). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody. In some embodiments, the heterodimeric antibody does not bind CD3.

In some embodiments, the first monomer includes from N- to C-terminus, VH1-CH1-hinge-CH2-CH3. In certain embodiments, the second monomer includes from N- to C-terminus VH2-scFv linker-VL2-linker-CH2-CH3, wherein VH2-scFv linker-VL2 is an scFv. In other embodiments, the second monomer includes from N- to C-terminus: VL2-scFv linker-VL2-linker-CH2-CH3, wherein VL2-scFv linker-VH2 is an scFv.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a BCMA antigen binding domain. In some embodiments, the first binding domain is any one of the BCMA ABDs described herein. In certain embodiments, the second binding domain is any one of the BCMA ABDS described herein. In some embodiments, the 1+1 Fab-scFv-Fc format antibody does not include a CD3 binding domain. Any one of the BCMA ABDs provided herein can be included in the 1+1 Fab-scFv-Fc format antibody, including one of the following BCMA ABDs: SIR4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15) or a variant thereof.

In some embodiments, the first and second Fc domains of the 1+1 Fab-scFv-Fc format antibody are variant Fc domains that include heterodimerization skew variants as disclosed herein (see, e.g., FIGS. 23 and 29). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C, wherein numbering is according to EU numbering. In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants as disclosed herein (see, e.g., FIG. 25). In some embodiments, each of the first and second variant Fc domains include ablation variants E233P/L234V/L235A/G236/S267K, wherein numbering is according to EU numbering.

In some embodiments, the constant domain (CH1-hinge-CH2-CH3) of the first monomer includes pI variants as disclosed herein (see, e.g., FIG. 24). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (see, e.g., FIG. 27). In some embodiments, the 1+1 Fab-scFv-Fc format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker. In some embodiments, the 1+1 Fab-scFv-Fc format antibody provided herein includes FcRn variants M428L/N434S, wherein numbering is according to EU numbering.

### 2. 2+1 Fab₂-scFv-Fc Format (Central-scFv Format)

One heterodimeric antibody format that finds particular use in the subject bispecific antibodies provided herein is the 2+1 Fab₂-scFv-Fc format (also referred to as “central-scFv format”) shown in FIG. 22B. This antibody format includes three antigen binding domains: two Fab portions and an scFv that is inserted between the VH-CH1 and CH2-CH3 regions of one of the monomers. In some embodiments of this format, the Fab portions each bind BCMA. In some embodiments, the “extra” scFv domain binds BCMA. In some embodiments, the 2+1 Fab₂-scFv-Fc format antibody is a trivalent antibody. In some embodiments, the heterodimeric antibody does not bind CD3.

In some embodiments of the 2+1 Fab₂-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 27. This embodiment further utilizes a common light chain that includes a variable light domain (VL1) and a constant light domain (CL). The common light chain associates with the VH1-CH1 of the first and second monomers to form two identical Fabs.

In some embodiments, the identical Fabs each bind BCMA. In some embodiments, the scFv binds BCMA. In some embodiments, the 2+1 Fab₂-scFv-Fc format antibody, none of the Fabs or scFvs bind CD3. Any one of the BCMA ABD provided herein can be included in the 2+1 Fab₂-scFv-Fc format antibody, including one of the following BCMA ABDs: S1R4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15) or a variant thereof.

In some embodiments, the first monomer includes from N- to C-terminus, VH1-CH1-hinge-CH2-CH3. In certain embodiments, the second monomer includes from N- to C-terminus VH1-CH1-first domain linker-VH2-scFv linker-VL2-second domain linker-CH2-CH3, wherein VH2-scFv linker-VL2 is an scFv. In other embodiments, the second monomer includes from N- to C-terminus: VH1-CH1-first linker-VL2-scFv linker-VL2-second linker-CH2-CH3, wherein VL2-scFv linker-VL2 is an scFv.

As for many of the embodiments herein, these constructs can include skew variants, pI variants, ablation variants, additional Fc variants, etc. as desired and described herein.

In some embodiments, the first and second Fc domains of the 2+1 Fab₂-scFv-Fc format antibody are variant Fc domains that include heterodimerization skew variants as disclosed herein (e.g., a set of amino acid substitutions as shown in FIGS. 23 and 29). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 25). In some embodiments, each of the first and second variant Fc domains include ablation variants E233P/L234V/L235A/G236/S267K, wherein numbering is according to EU numbering.

In some embodiments, the constant domain (CH1-hinge-CH2-CH3) of the first monomer includes pI variants (including those shown in FIG. 24). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab₂-scFv-Fc format antibody provided herein includes a charged scFv linker (including those shown in FIG. 26). In some embodiments, the 2+1 Fab₂-scFv-Fc format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab₂-scFv-Fc format antibody provided herein includes a (GKPGS)₄ charged scFv linker. In some embodiments, the 2+1 Fab₂-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

3. 2+1 mAb-scFv Format

One heterodimeric antibody format that finds particular use in the subject bispecific antibodies provided herein is the 2+1 mAb-scFv format shown in FIG. 22C. This antibody format includes three antigen binding domains: two Fab portions and an scFv that is attached to the C-terminal of one of the monomers. In some embodiments of this format, the Fab portions each bind BCMA, (e.g., human BCMA). In some embodiments, the “extra” scFv domain binds BCMA, (e.g., human BCMA).

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In these embodiments, the VH1-VL1 each form a first ABD and the VH2-VL2 form a second ABD. In some embodiments, the first ABD and/or the second ABD binds human BCMA.

In some embodiments, the first and second Fc domains of the 2+1 mAb-scFv format antibody are variant Fc domains that include heterodimerization skew variants (e.g., a set of amino acid substitutions as shown in FIGS. 23 and 29). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 25). In some embodiments, each of the first and second variant Fc domains include ablation variants E233P/L234V/L235A/G236/S267K, wherein numbering is according to EU numbering.

In some embodiments, the constant domain (CH1-hinge-CH2-CH3) of the first monomer includes pI variants (including those shown in FIG. 24). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 26). In some embodiments, the 2+1 mAb-scFv format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ charged scFv linker. In some embodiments, 2+1 mAb-scFv format antibody provided herein includes FcRn variants M428L/N434S, wherein numbering is according to EU numbering.

In some embodiments, the scFv of the second monomer of the 2+1 mAb-scFv format antibody binds BCMA. In some embodiments, the first and second monomer and the VL1 of the light chain each form binding domains that bind BCMA. Any suitable CD28 binding domain can be included in subject 2+1 mAb-scFv format antibody, including any of the BCMA binding domains provided herein. In some embodiments, the BCMA binding domain is one of the following BCMA ABDs: S1R4_10corr[BCMA]_HIL1, S1R5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15) or a variant thereof.

### 4. Monospecific, Monoclonal Antibodies

As will be appreciated by those in the art, the novel Fv sequences outlined herein can also be used in both monospecific antibodies (e.g., “traditional monoclonal antibodies”) or non-heterodimeric bispecific formats. Accordingly, the present invention provides monospecific antibodies comprising the 6 CDRs and/or the VH and VL sequences from the figures, generally with IgG1, IgG2, IgG3 or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody. In some embodiments, the monospecific antibody includes a monomer having, for N- to C-terminus, VH-CH1-hinge-CH2-CH3; and a light chain that includes a VL-CL.

In some embodiments, the monospecific antibody is an anti-BCMA monospecific antibody. In certain embodiments, the monospecific anti-BCMA antibody includes the 6 CDRs of any of the following BCMA ABDs: S1R4_10corr[BCMA]_HIL1, SIR5_125 [BCMA]_HIL1, 1A1 [BCMA]_HIL1, 1B4 [BCMA] _H1.1_L1, 1B3 [BCMA]_HIL1, 5F2 [BCMA]_HIL1, and 6E3 [BCMA]_HIL1 (FIGS. 5, 6, and 11-15) or a variant thereof.

### V. Recombinant Methods and Compositions

In another aspect, provided herein are nucleic acid compositions encoding the BCMA antigen binding domains and anti-BCMA antibodies provided herein.

As will be appreciated by those in the art, the nucleic acid compositions will depend on the format and scaffold of the heterodimeric protein. Thus, for example, when the format requires three amino acid sequences, such as for the 1+1 Fab-scFv-Fc or 2+1 Fab₂-scFv-Fc formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depends on the host cells used to produce the heterodimeric antibodies of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The antibodies and ABDs provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VI. Biological and Biochemical Functionality of the Anti-BCMA Antibodies

Generally, the bispecific anti-BCMA antibodies described herein are administered to patients with a BCMA-associated disease, disorder, or condition (e.g., a BCMA associated cancer), and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### VII. Antibody Compositions for In Vivo Administration

Formulations of the antibodies used in accordance with the present invention are prepared for storage by mixing an antibody having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### VIII. Therapeutic Uses

In another aspect, provided herein are methods for the treatment of a BCMA-associated disease, disorder, or condition (e.g., a BCMA associated cancer).

The antibodies provided herein administered to a subject, in accord with known methods, such as intravenous administration as a bolus, intravenous push or by intravenous infusion over a period of time. In preferred embodiments, the anti-BCMA antibody provided herein is administered by intravenous infusion.

In the methods of the invention, therapy is used to provide a positive therapeutic response with respect to a disease, disorder or condition described herein. Positive therapeutic responses in any given disease, disorder or condition can be determined by standardized response criteria specific to that disease or condition.

In addition to these positive therapeutic responses, the subject undergoing therapy may experience the beneficial effect of an improvement in the symptoms associated with the disease.

Treatment according to the present invention includes a “therapeutically effective amount” of the anti-BCMA antibody used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The efficient dosages and the dosage regimens for the anti-BCMA antibodies described herein depend on the disease or condition to be treated and may be determined by the persons skilled in the art.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are known in the art and outlined in US Publications 2015/0307629, 2014/0288275 and WO2014/145806, all of which are expressly incorporated by reference in their entirety and particularly for the techniques outlined therein.

### Example 1: Novel Phage-Derived BCMA Binding Domains

In-house de novo phage libraries were panned for novel BCMA binding domains. The variable heavy (VH) and variable light (VL) domain amino acid sequences for phage-derived clones SIR4_10corr and SIR5_125 are depicted in FIGS. 5 and 6, respectively.

### 1A: Production

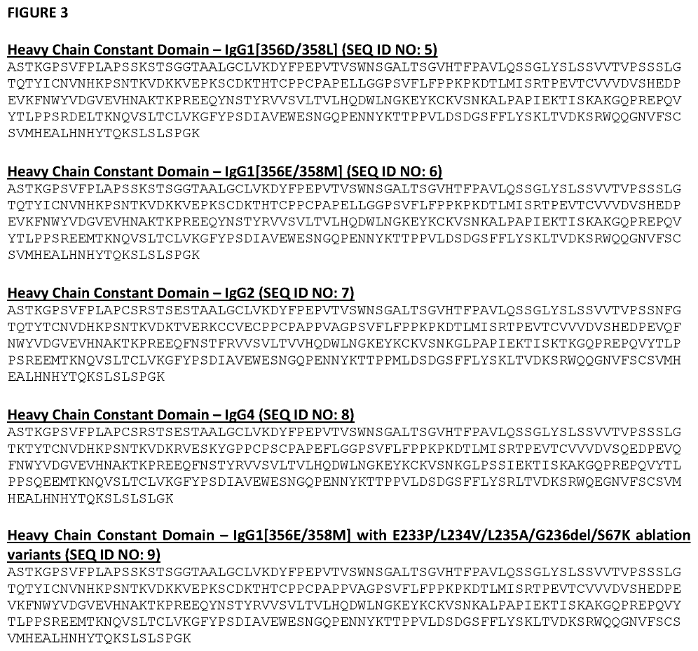

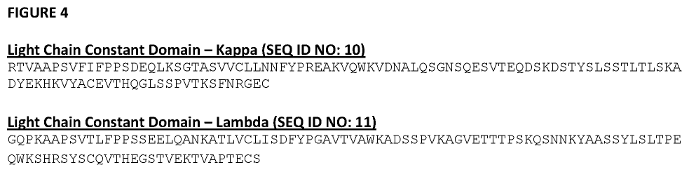

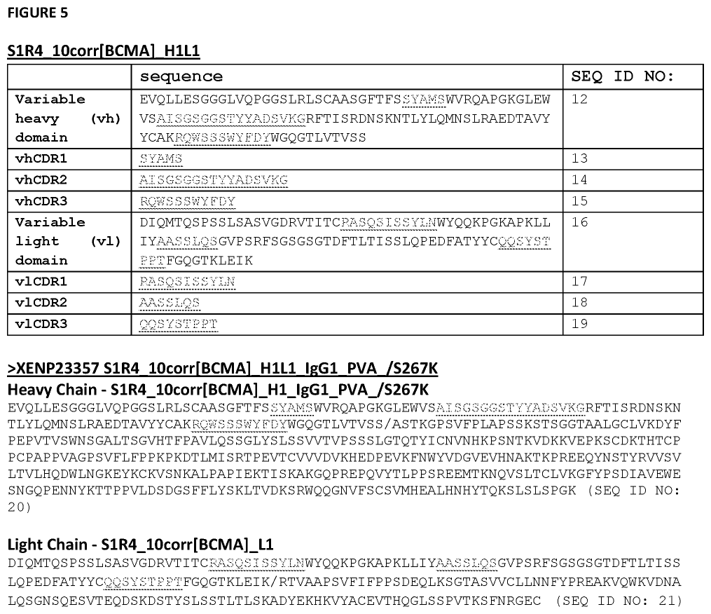

The phage-derived clones were formatted as bivalent monospecific mAbs, sequences for which are depicted in FIGS. 5 and 6. Plasmids containing the variable heavy and variable light domains of select clones were constructed by Gibson assembly and subcloned into pTT5 expression vectors containing the coding sequence for the IgG1 constant regions (with E233P/L234V/L235A/G236del/S67K ablation variants-see SEQ ID NO: 9; FIG. 3) and the kappa light constant region (see SEQ ID NO: 10; FIG. 4). DNA was transfected in HEK293E for expression and resulting bivalent mAbs were purified from the supernatant using protein A chromatography. XENP23357 was produced and purified at a yield of 135.2 mg/L and analyzed electrophoretically via CEF using LabChip and for aggregates via analytical size-exclusion chromatography, data for which are depicted in FIG. 7.

The phage-derived clones were also formatted as CD3 bispecific antibodies (bsAbs) and produced as generally described in PCT/US2015/062772 to further characterize for binding and activity as described below.

### 1B: BCMA Binding Affinity

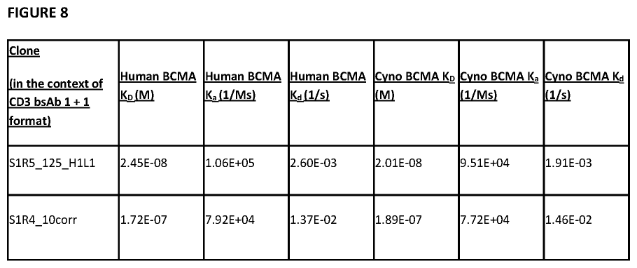

In this experiment, CD3 bsAbs in a 1+1 format having monovalent binding for CD3 and monovalent binding for BCMA were used. Monovalent KD values were obtained using Octet. HIS1K sensors were used to capture his-tagged human or cyno BCMA, and sensors were dipped into each bsAb at a range of concentrations. The resulting dissociation constant are depicted in FIG. 8.

### 1C: Cell Binding

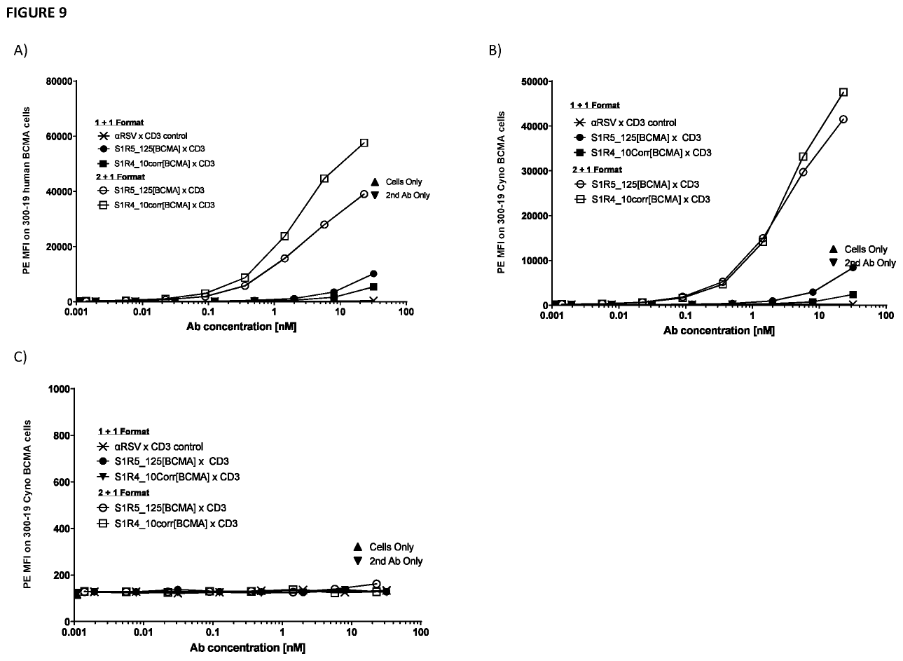

Next, binding to BCMA⁺ cells were investigated. In this experiment, CD3 bsAbs in the 1+1 format having monovalent binding for CD3 and monovalent binding for BCMA and in the 2+1 format having monovalent binding for CD3 and bivalent binding for BCMA were used. 300-19 cells were cultured and transfected to express either cyno BCMA or human BCMA. Parental 300-19 cells, along with the transfected 300-19 cells expressing either cyno BCMA or human BCMA, were seeded into a plate at 200K per well. All three cell types were then mixed with bsAbs at the range of concentrations indicated in the figure and incubated on ice for 1 hour. Next, samples were stained with PE anti-human IgG Fcγ (Jackson ImmunoResearch) at 1 μg/ml for another hour on ice. Samples were washed with PBS and fixed in PBS with 1% PFA before analysis on a FACS Canto 2 cytometer. Data are depicted in FIG. 9.

### 1D: In Vitro Activity

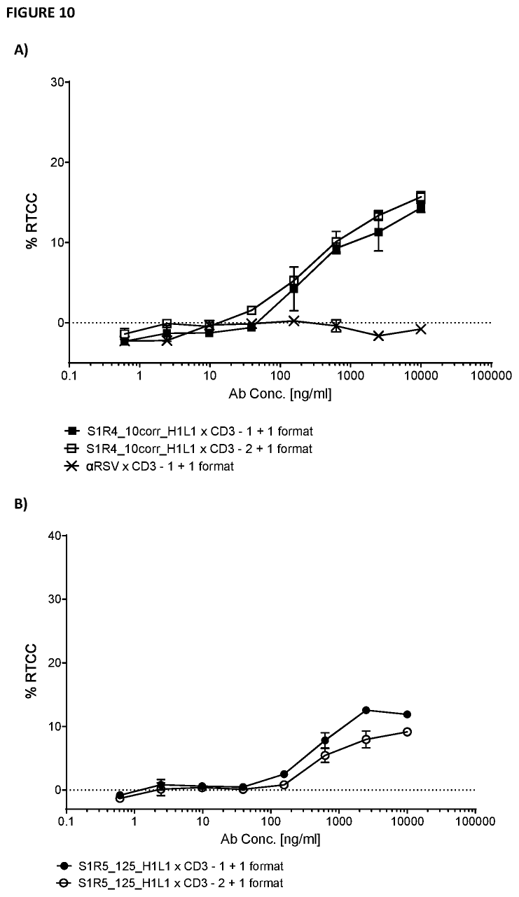

Finally, killing of RPMI8226 (BCMA⁺) cells were investigated. As above, CD3 bsAbs in the 1+1 format and 2+1 format were used. Additionally, an RSV×CD3 bsAb was used as control. Redirected T cell cytotoxicity (RTCC) was determined using CytoTox-ONE™ Homogeneous Membrane Integrity Assay (Promega, Madison, Wis.) to measure lactate dehydrogenase levels according to manufacturer's instructions and data was acquired on a Wallac Victor2 Microplate Reader (PerkinElmer, Waltham, Mass.). The data depicted in FIG. 10 show that each of the CD3 bsAbs based on phage-derived anti-BCMA clones S1R4_10corr or SIR1_125 were able to dose-dependently induce cell kill.

### Example 2: Novel Hybridoma-derived BCMA Binding Domains

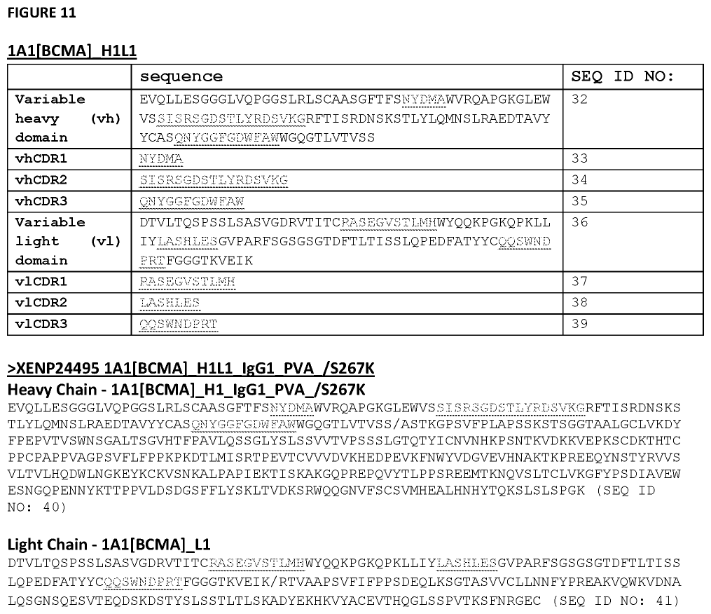

Additional novel BCMA binding domains were generated by rat hybridoma technology and humanized using string content optimization (see, e.g., U.S. Pat. No. 7,657,380). The variable heavy (V_{H}) and variable light (V_{L}) domain amino acid sequences for humanized hybridoma-derived clones 1A1, 1B4, 1B3, 4F2, and 6E3 are depicted in FIGS. 11-15.

### 2A: Production

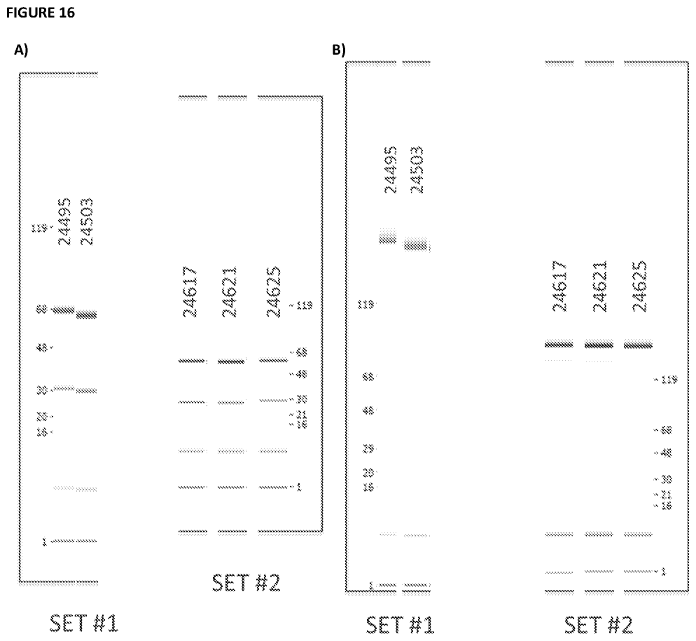

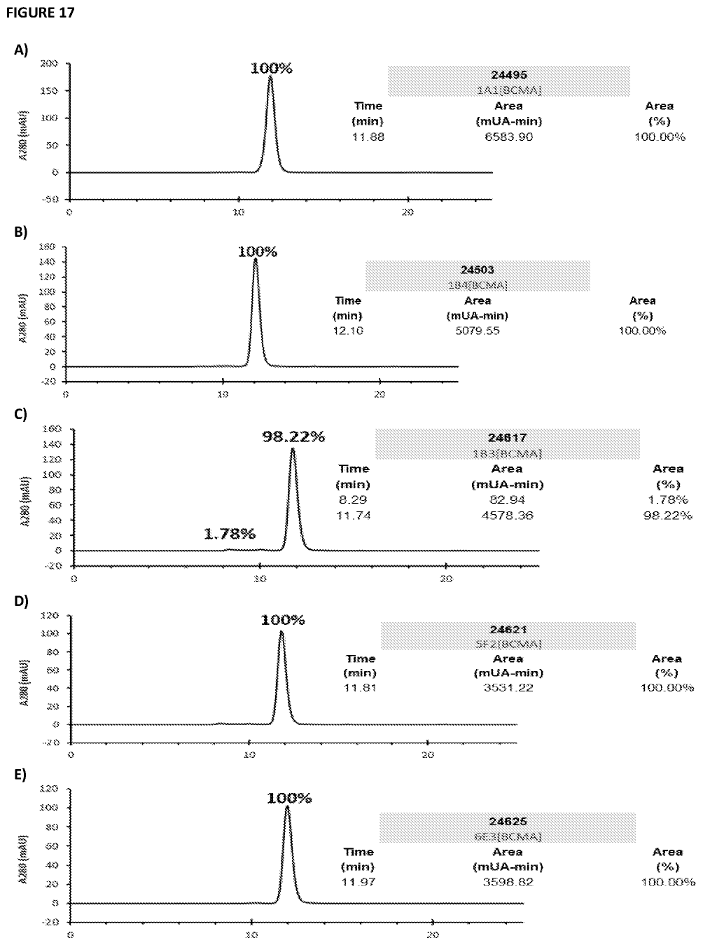

The hybridoma-derived clones were formatted as bivalent monospecific mAbs, sequences for which are depicted in FIGS. 11-15. Plasmids containing the variable heavy and variable light domains of select clones were constructed by Gibson assembly and subcloned into a pTT5 expression vector containing the coding sequence for the IgG1 constant regions (with E233P/L234V/L235A/G236del/S67K ablation variants-see SEQ ID NO: 9; FIG. 3) and the kappa light constant region (see SEQ ID NO: 10; FIG. 4). DNA was transfected in HEK293E for expression and resulting bivalent mAbs were purified from the supernatant using protein A chromatography. XENP24495 (1A1), XENP24503 (1B4), XENP24617 (1B3), XENP24621 (5F2), and XENP24625 (6E3) were produced and purified at yields of 66.49 mg/L, 124.37 mg/L, 45.04 mg/L, 58.58 mg/L, and 76.43 mg/L respectively. The purified mAbs were analyzed electrophoretically via CEF using LabChip and for aggregates via analytical size-exclusion chromatography, data for which are depicted in FIGS. 16-17.

The hybridoma-derived clones were also formatted as CD3 bispecific antibodies (bsAbs) and produced as generally described in PCT/US2015/062772 to further characterize for binding and activity as described below.

### 2B: BCMA Binding Affinity

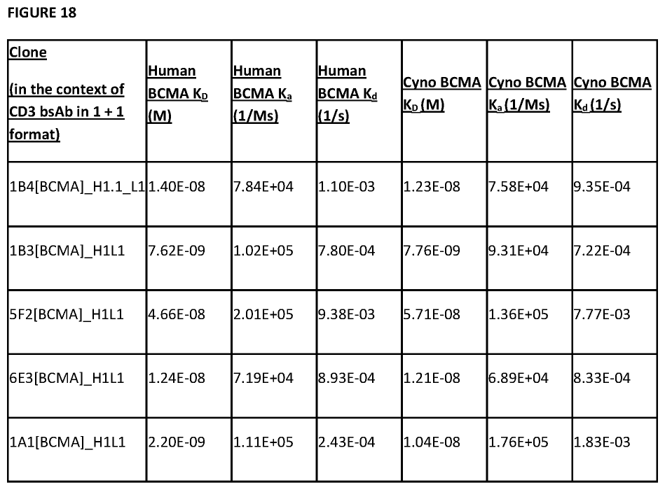

In this experiment, CD3 bsAbs in a 1+1 format having monovalent binding for CD3 and monovalent binding for BCMA were used. Monovalent KD values were obtained using Octet. HIS1K sensors were used to capture his-tagged human or cyno BCMA, and sensors were dipped into each bsAb at a range of concentrations. The resulting dissociation constant are depicted in FIG. 18.

### 2C: Cell Binding

Next, binding to BCMA⁺ cells were investigated. In this experiment, bivalent BCMA mAbs were used. 300-19 cells were cultured and transfected to express either cyno BCMA or human BCMA. Parental 300-19 cells, along with the transfected 300-19 cells expressing either cyno BCMA or human BCMA, were seeded into a plate at 200K per well. All three cell types were then mixed with mAbs at the range of concentrations indicated in the figure and incubated on ice for 1 hour. Next, samples were stained with PE anti-human IgG Fcγ (Jackson ImmunoResearch) at 1 μg/ml for another hour on ice. Samples were washed with PBS and fixed in PBS with 1% PFA before analysis on a FACS Canto 2 cytometer. Data are depicted in FIG. 19.

### 2D: In Vitro Activity

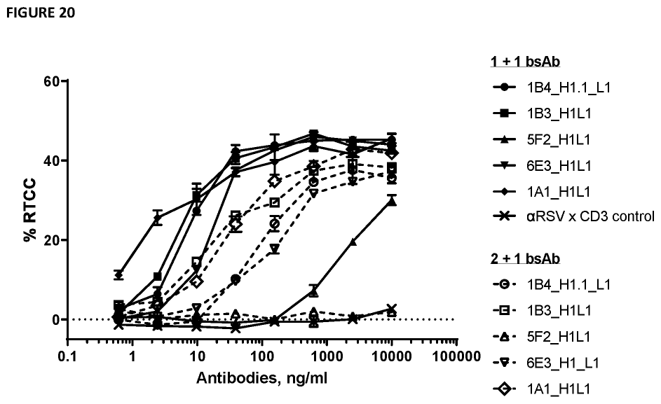

Killing of RPMI8226 (BCMA⁺) cells were investigated. As above, CD3 bsAbs in the 1+1 format and 2+1 format were used. Additionally, an RSV×CD3 bsAb was used as control. Redirected T cell cytotoxicity (RTCC) was determined using CytoTox-ONE™ Homogeneous Membrane Integrity Assay (Promega, Madison, Wis.) to measure lactate dehydrogenase levels according to manufacturer's instructions and data was acquired on a Wallac Victor2 Microplate Reader (PerkinElmer, Waltham, Mass.). The data depicted in FIG. 20 show that each of the CD3 bsAbs based on each of the hybridoma-derived anti-BCMA clones were able to dose-dependently induce cell kill in contrast to the control bsAb having an RSV binding domain.

### 2E: Pharmacokinetic

Finally, the pharmacokinetics of the hybridoma-derived binding domains, in the context of CD3 bsAbs in the 1+1 format, to investigate stability of the molecules. For each bsAb investigated, 5 mice were dosed at 2 mg/kg on Day 0. After 1 hour, 50 μl total blood was collected from each mouse. This blood collection was repeated on days 1, 3, 7, 10 & 14. Mouse serum was assayed by ELISA and analyzed using an Envision plate reader in order to determine half-life, data for which are depicted in FIG. 21.

What is claimed is:

1. A composition comprising an anti-BCMA antigen binding domain comprising a variable light domain and a variable heavy domain selected from the following sets of variable light domains and variable heavy domains:
a) a variable light domain comprising a vlCDR1 having an amino acid of SEQ ID NO: 17, a vlCDR2 having an amino acid of SEQ ID NO: 18, and a vlCDR3 having an amino acid of SEQ ID NO: 19 and a variable heavy domain comprising a vhCDR1 having an amino acid of SEQ ID NO: 13, a vhCDR2 having an amino acid of SEQ ID NO: 14 and a vhCDR3 having an amino acid of SEQ ID NO: 15;
b) a variable light domain comprising a vlCDR1 having an amino acid of SEQ ID NO: 27, a vlCDR2 having an amino acid of SEQ ID NO: 28, and a vlCDR3 having an amino acid of SEQ ID NO: 29 and a variable heavy domain comprising a vhCDR1 having an amino acid of SEQ ID NO: 23, a vhCDR2 having an amino acid of SEQ ID NO: 24 and a vhCDR3 having an amino acid of SEQ ID NO: 25;
c) a variable light domain comprising a vlCDR1 having an amino acid of SEQ ID NO: 37, a vlCDR2 having an amino acid of SEQ ID NO: 38, and a vlCDR3 having an amino acid of SEQ ID NO: 39 and a variable heavy domain comprising a vhCDR1 having an amino acid of SEQ ID NO: 33, a vhCDR2 having an amino acid of SEQ ID NO: 34 and a vhCDR3 having an amino acid of SEQ ID NO: 35;
d) a variable light domain comprising a vlCDR1 having an amino acid of SEQ ID NO: 47, a vlCDR2 having an amino acid of SEQ ID NO: 48, and a vlCDR3 having an amino acid of SEQ ID NO: 49 and a variable heavy domain comprising a vhCDR1 having an amino acid of SEQ ID NO: 43, a vhCDR2 having an amino acid of SEQ ID NO: 44 and a vhCDR3 having an amino acid of SEQ ID NO: 45;
e) a variable light domain comprising a vlCDR1 having an amino acid of SEQ ID NO: 57, a vlCDR2 having an amino acid of SEQ ID NO: 58, and a vlCDR3 having an amino acid of SEQ ID NO: 59 and a variable heavy domain comprising a vhCDR1 having an amino acid of SEQ ID NO: 53, a vhCDR2 having an amino acid of SEQ ID NO: 54 and a vhCDR3 having an amino acid of SEQ ID NO: 55;
f) a variable light domain comprising a vlCDR1 having an amino acid of SEQ ID NO: 67, a vlCDR2 having an amino acid of SEQ ID NO: 68, and a vlCDR3 having an amino acid of SEQ ID NO: 69 and a variable heavy domain comprising a vhCDR1 having an amino acid of SEQ ID NO: 63, a vhCDR2 having an amino acid of SEQ ID NO: 64 and a vhCDR3 having an amino acid of SEQ ID NO: 65; and
g) a variable light domain comprising a vlCDR1 having an amino acid of SEQ ID NO: 77, a vlCDR2 having an amino acid of SEQ ID NO: 78, and a vlCDR3 having an amino acid of SEQ ID NO: 79 and a variable heavy domain comprising a vhCDR1 having an amino acid of SEQ ID NO: 73, a vhCDR2 having an amino acid of SEQ ID NO: 74 and a vhCDR3 having an amino acid of SEQ ID NO: 75.

2. A composition comprising an anti-BCMA antigen binding domain comprising a set of a variable light domain and a variable heavy domain selected from the following sets of variable light domains and variable heavy domains:
a) a variable light domain comprising a vlCDR1, vlCDR2, and vlCDR3 of a variable light domain having the amino acid of SEQ ID NO: 16 and a variable heavy domain comprising a vhCDR1, vhCDR2, and vhCDR3 of a variable heavy domain having the amino acid sequence of SEQ ID NO: 12;
b) a variable light domain comprising a vlCDR1, vlCDR2, and vlCDR3 of a variable light domain having the amino acid of SEQ ID NO: 26 and a variable heavy domain comprising a vhCDR1, vhCDR2, and vhCDR3 of a variable heavy domain having the amino acid sequence of SEQ ID NO: 22;
c) a variable light domain comprising a vlCDR1, vlCDR2, and vlCDR3 of a variable light domain having the amino acid of SEQ ID NO: 36 and a variable heavy domain comprising a vhCDR1, vhCDR2, and vhCDR3 of a variable heavy domain having the amino acid sequence of SEQ ID NO: 32;
d) a variable light domain comprising a vlCDR1, vlCDR2, and vlCDR3 of a variable light domain having the amino acid of SEQ ID NO: 46 and a variable heavy domain comprising a vhCDR1, vhCDR2, and vhCDR3 of a variable heavy domain having the amino acid sequence of SEQ ID NO: 42;
e) a variable light domain comprising a vlCDR1, vlCDR2, and vlCDR3 of a variable light domain having the amino acid of SEQ ID NO: 56 and a variable heavy domain comprising a vhCDR1, vhCDR2, and vhCDR3 of a variable heavy domain having the amino acid sequence of SEQ ID NO: 52;
f) a variable light domain comprising a vlCDR1, vlCDR2, and vlCDR3 of a variable light domain having the amino acid of SEQ ID NO: 66 and a variable heavy domain comprising a vhCDR1, vhCDR2, and vhCDR3 of a variable heavy domain having the amino acid sequence of SEQ ID NO: 62; and
g) a variable light domain comprising a vlCDR1, vlCDR2, and vlCDR3 of a variable light domain having the amino acid of SEQ ID NO: 76 and a variable heavy domain comprising a vhCDR1, vhCDR2, and vhCDR3 of a variable heavy domain having the amino acid sequence of SEQ ID NO: 72.

3. A composition comprising an anti-BCMA antigen binding domain comprising a set of a variable light domain and a variable heavy domain selected from the following sets of variable light domains and variable heavy domains:
a) a variable light domain having an amino acid of SEQ ID NO: 16 and a variable heavy domain having an amino acid of SEQ ID NO: 12;
b) a variable light domain having an amino acid of SEQ ID NO: 26 and a variable heavy domain having an amino acid of SEQ ID NO: 22;
c) a variable light domain having an amino acid of SEQ ID NO: 36 and a variable heavy domain having an amino acid of SEQ ID NO: 32;
d) a variable light domain having an amino acid of SEQ ID NO: 46 and a variable heavy domain having an amino acid of SEQ ID NO: 42;
e) a variable light domain having an amino acid of SEQ ID NO: 56 and a variable heavy domain having an amino acid of SEQ ID NO: 52;
f) a variable light domain having an amino acid of SEQ ID NO: 66 and a variable heavy domain having an amino acid of SEQ ID NO: 62; and
g) a variable light domain having an amino acid of SEQ ID NO: 76 and a variable heavy domain having an amino acid of SEQ ID NO: 72.

4. A nucleic acid composition comprising:
a) a first nucleic acid encoding a variable heavy domain according to claim 1, 2, or 3; and
b) a second nucleic acid encoding a variable light domain according to claim 1, 2 or 3, respectively.

5. An expression vector composition comprising:
a) a first expression vector comprising the first nucleic acid of claim 4; and
b) a second expression vector comprising the second nucleic acid of claim 4.

6. A host cell comprising the expression vector composition of claim 5.

7. A method of making a BCMA binding domain comprising culturing the host cell according to claim 6 under conditions wherein said BCMA binding domain is expressed and recovering said BCMA binding domain.
